(19) United States
(12) Patent Application Publication
(10) Pub. No.: US 2022/0125867 A1
(43) Pub. Date: Apr. 28, 2022

(54) CANNABINOID CONTAINING COMPOSITION, METHODS OF PREPARATION AND USE THEREOF

(71) Applicant: CANNASOUL ANALYTICS LTD., Caesarea Industrial Zone (IL)

(72) Inventors: Shmuel Mandel, Atlit (IL); Mili Cravchick, Pardes Hanna-Karkur (IL); Rotem Perry-Feigenbaum, Bazra (IL); David Meiri, Tzur Moshe (IL)

(21) Appl. No.: 17/428,968

(22) PCT Filed: Feb. 6, 2020
(86) PCT No.: PCT/IL2020/050143

§ 371 Date: Aug. 5, 2021

Related U.S. Application Data
(60) Provisional application No. 62/893,527, filed on Aug. 29, 2019.
(60) Provisional application No. 62/876,715, filed on Jul. 21, 2019.
(60) Provisional application No. 62/802,233, filed on Feb. 7, 2019.

(51) Int. Cl. A61K 36/185; A61K 47/40; A61K 47/26; A61K 31/05; A61K 31/352

(52) U.S. Cl. CPC A61K 36/185; A61K 47/40; A61K 47/26; A61K 31/05; A61K 2236/51; A61K 2236/37; A61K 2236/35; A61K 2236/333; A61K 31/352

(57) ABSTRACT

Solid compositions including a plurality of cannabinoids and methods of preparing such solid compositions are provided. The plurality of cannabinoids in the solid compositions are characterized by a chemical signature which is substantially identical to the chemical signature of cannabinoids in an extract of the Cannabis plant thereby exerting the entourage effect.

### FIELD OF THE INVENTION

The present invention provides a solid composition, preferably in the form of a powder, comprising a plurality of cannabinoids having a chemical signature which is substantially identical to their chemical signature in an extract of the Cannabis plant. The present invention further provides methods of preparing said composition and use thereof in a variety of applications.

### BACKGROUND OF THE INVENTION

Cannabis is a genus of flowering plants in the family Cannabaceae which includes three species, namely Cannabis sativa, Cannabis indica, and Cannabis ruderalis. It is considered as the primary, almost exclusive, botanical source of phytocannabinoids. There are more than 100 different phytocannabinoids that have been identified in Cannabis plants. These cannabinoids are capable of acting on a cannabinoid receptor thereby modulating the release of neurotransmitters. Some cannabinoids are known to exhibit a physiological effect when administered to an organism thereby being useful in a variety of medical applications including treatment of nausea due to chemotherapy, spasticity, and neuropathic pain. The exact identity and relative amounts of each cannabinoid in a given Cannabis preparation depends primarily on the variety of the Cannabis plant, and to some extent, on the conditions in which the plant was grown and harvested. As a result, the physiological effect of a Cannabis preparation may vary.

While single cannabinoids have been shown to exert biological activity, Cannabis synergy, also known as the “entourage effect”, in which a variety of “minor cannabinoids” and Cannabis terpenoids markedly increase the activity of the primary endogenous cannabinoids has long been recognized (Ben-Shabat et al., 1998, Eur. J. Pharmacol. 353:23-31, McPartland, 2001, J. Cannabis Ther. 1:103-132). Accordingly, botanical drug preparations which preserve the Cannabis synergy are desirable in order to obtain high medical efficacy. U.S. 2018/0369192 describes enriched or non-naturally occurring compositions which contain defined concentrations of one or more Cannabis-derived chemical compounds, such as terpenes and cannabinoids that have a distinctive characteristic that mimics that of a Cannabis plant matter or a product thereof.

The highly lipophilic nature of cannabinoids renders their processing into bioavailable drug preparations challenging. Oily preparations for oral administration are considered to afford less than 15% bioavailability due to the massive degradation of cannabinoids in the liver prior to reaching the bloodstream. While smoking provides relatively high bioavailability of the Cannabis components, it is not suitable for all patients, especially kids, the elderly population and patients suffering from pulmonary disorders.

U.S. 2018/0369191 describes a blended and processed hybrid Cannabis compound for relieving symptoms of at least one of pain, inflammation, or arthritis in a canine in need thereof, the hybrid Cannabis compound comprising: a Cannabis component comprising a hybrid of at least two of Cannabis sativa, Cannabis indica, and Cannabis ruderalis; and at least one additive, wherein the Cannabis component comprises from about 0.01 to about 25% tetrahydrocannabinol (THC) and from about 0.1 to about 26% cannabidiol (CBD).

U.S. 2018/0263913 describes a composition comprising one or more cannabinoids mixed with starch acetate, cyclodextrin and at least one additional excipient wherein the composition provides both fast release and sustained release of the cannabinoids, wherein the composition is in the form of a multi-layer tablet.

U.S. 2018/0193304 describes a formulation, comprising: at least 30% tetrahydrocannabinol; at least 8% cannabidiol; and at least 10% β-caryophyllene.

U.S. 2018/0184705 describes methods for creating concentrated plant material solution, including combining ethanol-soluble, water-insoluble concentrated plant material with ethanol to define mixtures and reducing the amount of ethanol in the mixtures until the mixtures have viscosities compatible for use with electronic cigarettes.

U.S. 2018/0125980 describes a cannabinoid composition, comprising: at least one cannabinoid, and an excipient including α-tocopherol.

U.S. 2016/0151328 describes a liquid composition comprising, in combination: at least two terpenes; and at least one enhancer selected from the group consisting of triacetin, dipropylene glycol, isophytol and phytol. The composition optionally includes cannabinoids.

U.S. 2019/0030170 describes a clear ready-to-inject liquid formulation comprising BE7-3-cyclodextrin and at least one cannabinoid, the at least one cannabinoid is present in amount of 0.5-10 mg/mL.

U.S. 2018/0344688 describes a composition, comprising: a mixture including: an amount of a cannabinoid-cyclodextrin complex including a cannabinoid associated with a β-cyclodextrin; and an amount of a cannabinoid micelle.

U.S. 2018/0085308, U.S. 2018/0263953 and U.S. 2018/0263954 describe modified release pharmaceutical compositions comprising one or more natural or synthetic cannabinoids and one or more pharmaceutically acceptable excipients including a cyclodextrin.

U.S. 2018/0206518 describes a method of making Cannabis oil hydrophilic comprising the steps of: heating a base oil in the range of 120 to 220° F.; adding Cannabis oil to create a mixture; blending said mixture at a high speed and adding at least one emulsifying agent to said mixture while blending; and adding water to form the composition, wherein the Cannabis oil is hydrophilic and is soluble in water.

U.S. 2018/0200315 describes a cannabinoid emulsification comprising: at least one emulsifying agent selected from the group consisting of xanthan gum, guar gum, cyclodextrin, lecithin, carrageen, monoglycerides, natural emulsifiers and organic emulsifiers that are safe for ingestion by humans; an aqueous vehicle selected from the group consisting of coconut water, fruit juice, milk and water; a base oil; Cannabis oil; and caffeine; wherein the emulsification modifies said Cannabis oil such that it is hydrophilic and soluble in said aqueous vehicle.

U.S. 2018/0117161 describes a cannabinoid infused food product comprising: (a) a therapeutically effective amount of the cannabinoid; (b) a bioavailability enhancing agent, wherein the bioavailability enhancing agent enhances the bioavailability of the cannabinoid; (c) a starch, wherein the starch is selected from the group consisting of tapioca starch, corn starch, potato starch, gelatin, dextrin, cyclodextrin, oxidized starch, starch ester, starch ether, crosslinked starch, a starch, octenyl succinate ester, and processed starch obtained by treating a starch by an acid, heat, or enzyme; and (d) a food product, wherein the food product is selected from the group consisting of tea leaves, coffee beans, cocoa powder, meats, fish, fruits, vegetables, dairy products, legumes, pastas, breads, grains, seeds, nuts, spices, and herbs.

U.S. 2017/0232210 describes a method of manufacturing a flowable and dispersible powder, the method comprising: solubilizing a lipophilic substance in a terpene to form a mixture; adding at least one functional excipient to water to form an aqueous solution; dispersing the mixture into the aqueous solution using one or both of a homogenizer or an ultrasonic device to form a coarse emulsion; treating the coarse emulsion with one or both of the ultrasonic device or a high shear device to form a nanoemulsion; and spraying drying the nanoemulsion, thereby evaporating at least a portion of the terpene and substantially all of the water to form a dry powder formed from solid particles comprising the lipophilic substance.

U.S. 2009/0298929 describes a complex comprising: (a) β-cyclodextrin (b) a cannabinoid wherein said complex is an insoluble powder form.

U.S. 2007/0104741 describes an oral dosage form of cannabinoids comprising a pharmacologically active form of cannabinoids in a self-emulsifying system comprising an oily medium selected from the group consisting of triglycerides, mixed glycerides, free fatty acids having from C₆ to C₃₂ carbon atoms, and mixtures thereof; and a surfactant which promotes self-emulsification.

U.S. 2005/0153931 describes the use of a complex of a cyclodextrin selected from the group consisting of α-CD, β-CD and γ-CD and a cannabinoid selected from the classical cannabinoid-group consisting of cannabinol, tetrahydrocannabinol and cannabidiol for the preparation of a pharmaceutical composition for sublingual or buccal administration.

U.S. 2013/0089600 describes a stable, aqueous micelle suspension of one or more cannabinoids or cannabinoid analogues, wherein the stable aqueous micelle suspension of one or more cannabinoids or cannabinoid analogues does not comprise phospholipids and cholesterol.

U.S. 2016/0367522 describes a method of preparing a granulate having a mass weighted average diameter of 50-500 μm, comprising: (a) providing a lactose powder having a mass weighted average diameter of 32-250 μm; (b) granulating the lactose powder by combining the powder with a granulation liquid comprising a solution of 10-75 wt. % of cannabinoid with sucrose fatty acid mono-ester having a C₈-C₁₈ fatty acid residue in a C₁-C₃ alcohol organic solvent; and (c) removing the organic solvent by evaporation, wherein the granules comprise 40-99 wt. % lactose.

U.S. 2018/0271826 describes a dry powder comprising at least one cannabinoid, a polymer binding agent, a dispersing agent, and a bulking agent, and optionally an antioxidant, the dry powder formed by carbon dioxide-assisted nebulization of a solution comprising at least one cannabinoid, a polymer binding agent, a dispersing agent, and a bulking agent, and optionally an antioxidant and drying droplets formed by the nebulization in a flowing stream of gas to produce a dry powder, wherein the dry powder has an aerodynamic particle distribution effective for delivery of the dry powder by respiration into a lung of a patient.

U.S. 2017/0348276 describes a nasal pharmaceutical composition for topical application in the nasal cavity of a subject, said nasal pharmaceutical composition comprising: (a) a therapeutically effective amount of a cannabinoid; and (b) a pharmaceutically acceptable excipient, wherein the nasal pharmaceutical composition is a semi-solid or viscous liquid nasal pharmaceutical composition.

There remains an unmet need for compositions comprising a plurality of cannabinoids exhibiting the entourage effect that can be easily formulated in a variety of dosage forms with improved bioavailability.

### SUMMARY OF THE INVENTION

The present invention provides a method for preparing a solid cannabinoid composition in the form of a powder, the composition comprising a plurality of cannabinoids characterized by a chemical signature substantially identical to the chemical signature of the extract of Cannabis from which the composition is obtained. The present invention further provides a solid cannabinoid composition in the form of a powder which is suitable for use in a variety of applications, particularly medicinal applications.

The present invention is based, in part, on the surprising discovery of a robust and highly efficient method for preparing a solid powder composition comprising a plurality of organic compounds extracted from Cannabis, the plurality of organic compounds including a plurality of cannabinoids, which are present in the composition in relative amounts that are substantially identical to their relative amounts in the Cannabis extract. Accordingly, the synergy between different Cannabis constituents, known as the entourage effect, is maintained. Whereas the extract from the Cannabis plant is typically in the form of a highly-viscous crude oil that is difficult to process, the powder composition of the present invention is advantageously easily manipulated. It can therefore be used in a variety of analytical and pharmaceutical applications.

According to a first aspect, there is provided a method for preparing a solid cannabinoid composition in the form of a powder, the method comprising the steps of:

(i) providing a first solution comprising an extract of Cannabis comprising a plurality of cannabinoids dissolved in a first solvent, wherein said extract, when dried to a residual solvent of about 5,000 parts per million (ppm) or less, is a non-powder crude oil having a viscosity of at least 3,000 cps as determined at 40° C.;

(ii) adding a carrier to the first solution to obtain a mixture comprising said plurality of cannabinoids and carrier; and

(iii) removing at least 90% by weight of the solvent from the mixture, thereby obtaining a solid cannabinoid composition in the form of a powder,

wherein the plurality of cannabinoids in the solid cannabinoid composition has a chemical signature which is substantially identical to the chemical signature of the plurality of cannabinoids in the extract of Cannabis, and wherein the solid cannabinoid composition has a Hausner ratio of less than 1.59.

According to another aspect, there is provided a method for preparing a solid cannabinoid composition in the form of a powder, the method comprising the steps of:

(i) providing an extract of Cannabis comprising a plurality of cannabinoids, wherein said extract, when dried to a residual solvent of about 5,000 ppm or less, is a non-powder crude oil having a viscosity of at least 3,000 cps as determined at 40° C.;

(ii) dissolving said extract in a first solvent to obtain a first solution;

(iii) adding a carrier to the first solution to obtain a mixture comprising said plurality of cannabinoids and carrier; and

(iv) removing at least 90% by weight of the solvent from the mixture, thereby obtaining a solid cannabinoid composition in the form of a powder,

wherein the plurality of cannabinoids in the solid cannabinoid composition has a chemical signature which is substantially identical to the chemical signature of the plurality of cannabinoids in the extract of Cannabis, and wherein the solid cannabinoid composition has a Hausner ratio of less than 1.59.

In one embodiment, the extract of Cannabis is a non-powder crude oil having a residual solvent of about 5,000 ppm or less and a viscosity of at least 3,000 cps as determined at 40° C. In further embodiments, the viscosity of said dried extract of Cannabis is in the range of about 3,000 to about 2,000,000 cps as determined at 40° C., including each value within the specified range. In additional embodiments, the viscosity of said dried extract of Cannabis is in the range of about 5,000 to about 1,000,000 cps as determined at 40° C., including each value within the specified range. In other embodiments, the viscosity of said dried extract of Cannabis is in the range of about 10,000 to about 500,000 cps as determined at 40° C., including each value within the specified range.

In some embodiments, the extract of Cannabis is obtained from any species of the family Cannabaceae. In particular embodiments, the extract of Cannabis is obtained from Cannabaceae species selected from Cannabis sativa, Cannabis indica, Cannabis ruderalis, and a mixture or combination thereof. Each possibility represents a separate embodiment. In one embodiment, the extract of Cannabis is obtained from a single species of Cannabis. In other embodiments, the extract of Cannabis is obtained from hemp. In further embodiments, the extract of Cannabis is obtained using at least one of organic solvent extraction, carbon dioxide (dry ice) extraction, supercritical and subcritical carbon dioxide extraction, hydrocarbon extraction, rosin press, and a combination thereof. Each possibility represents a separate embodiment.

In various embodiments, the solvent used to dissolve the extract of Cannabis to obtain a first solution is an organic solvent selected from the group consisting of C₁-C₈ aliphatic alcohols, C₁-C₁₀ aliphatic hydrocarbons, C₆-C₁₀ aromatic hydrocarbons, C₂-C₈ aliphatic esters, C₂-C₈ aliphatic ketones, C₄-C₈ ethers, C₁-C₁₀ halo-substituted aliphatic hydrocarbons, C₂-C₈ aliphatic amides, and a mixture or combination thereof. Each possibility represents a separate embodiment. In some exemplary embodiments, the organic solvent is selected from the group consisting of ethanol, methanol, isopropyl alcohol, n-butanol, t-butyl alcohol, acetone, methyl ethyl ketone, toluene, benzene, hexane, cyclohexane, heptane, pentane, methyl acetate, ethyl acetate, t-butyl acetate, isopropyl acetate, diisopropyl ether, methyl t-butyl ether, tetrahydrofuran, dioxane, dichloromethane, dichloroethane, chloroform, carbon tetrachloride, tetrachloroethylene, tetrachloroethane, dimethylformamide, dimethylacetamide, and a mixture or combination thereof. Each possibility represents a separate embodiment.

In further embodiments, the carrier is dissolved or suspended in a second solvent which is then admixed with the first solution to obtain said mixture. In various embodiments, the solvent used to dissolve or suspend the carrier is a solvent that is miscible in the first solvent. In some exemplary embodiments, the solvent used to dissolve or suspend the carrier is an aqueous solvent. In other exemplary embodiments, the solvent used to dissolve or suspend the carrier is selected from the group consisting of water, acetone, ethanol, methanol, dimethyl formamide, DMSO, and a mixture or combination thereof. Each possibility represents a separate embodiment. In additional embodiments, the ratio between the first solvent and the second solvent is in the range of about 1:10 to about 10:1, including all iterations of ratios within the specified range. In further embodiments, the ratio between the first solvent and the second solvent is in the range of about 1:5 to about 5:1, including all iterations of ratios within the specified range. In other embodiments, the ratio between the first solvent and the second solvent is in the range of about 1:5 to about 1:1, including all iterations of ratios within the specified range. In particular embodiments, the ratio between the first solvent and the second solvent is in the range of about 1:2.

In certain embodiments, removal of the solvent is performed using at least one of the following techniques: evaporation, optionally at elevated temperatures and/or reduced pressures, freeze-drying (lyophilization), distillation, air drying, spray drying, fluid bed drying, or a combination thereof. Each possibility represents a separate embodiment. In currently preferred embodiments, removal of the solvent is performed using a combination of evaporation and lyophilization. It is contemplated that solvent removal is performed such that at least 90% by weight of the solvent is removed. In some embodiments, at least 95% by weight of the solvent is removed. In yet other embodiments, at least 97% by weight of solvent is removed.

In some embodiments, the solid composition disclosed herein has a characteristic fingerprint similar to that of the extract of Cannabis. It is contemplated that the composition disclosed herein comprises a plurality of cannabinoids (i.e. two or more cannabinoids) which are present in the Cannabis extract, said plurality of cannabinoids are in relative amounts which are substantially identical to their relative amounts in the Cannabis extract. In some embodiments, the plurality of cannabinoids is selected from the group consisting of cannabidivarinic acid (CBDVA), cannabidiolic acid (CBDA), cannabigerolic acid (CBGA), cannabidiol (CBD), cannabinol (CBN), cannabinolic acid (CBNA), tetrahydrocannabinol (THC), cannabichromene (CBC), cannabichromenic acid (CBCA), tetrahydrocannabinolic acid (THCA), cannabicitran, and a mixture or combination thereof. Each possibility represents a separate embodiment. In one embodiment, the plurality of cannabinoids comprises at least two of the aforementioned cannabinoids, for example 2, 3, 4, 5, 6, 7, 8, 9, 10, or all 11 cannabinoids. Each possibility represents a separate embodiment. In certain embodiments, the plurality of cannabinoids further comprises at least one of tetrahydrocannabivarin (THCV), cannabigerol (CBG), sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabigerolic acid monomethyl ether (CBGMA), cannabigerol monomethyl ether (CBGM), cannabicyclol (CBL), cannabicyclolic acid (CBLA), THCA-C4, THC-C4, tetrahydrocannabivarin carboxylic acid (THCVA), tetrahydrocannabivarin (THCV), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol (CBN), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, and a mixture or combination thereof. Each possibility represents a separate embodiment. In various embodiments, the solid composition disclosed herein further comprises other components extracted from a plant in the genus Cannabis, for example at least one of terpenes, terpenoids, flavonoids, nitrogenous compounds, amino acids, proteins, glycoproteins, sugars, hydrocarbons, fatty acids, esters, lactones, steroids, non-cannabinoid phenols, and a mixture or combination thereof. Each possibility represents a separate embodiment.

According to some embodiments of the invention, there is provided a solid cannabinoid composition in the form of a powder, comprising: a carrier; and a plurality of organic compounds extracted from Cannabis, wherein the plurality of organic compounds includes a plurality of cannabinoids characterized by a chemical signature which is substantially identical to their chemical signature in the extract of Cannabis, wherein the composition is obtained by the method disclosed herein. In certain embodiments, the composition has a Hausner ratio of less than 1.59, for example between 1.00 and 1.59, including each value within the specified range. In one embodiment, the composition has a Hausner ratio in the range of 1.46 to 1.59, including each value within the specified range. In another embodiment, the composition has a Hausner ratio in the range of 1.35 to 1.45, including each value within the specified range. In yet another embodiment, the composition has a Hausner ratio in the range of 1.26 to 1.34, including each value within the specified range. In other embodiments, the composition has a Hausner ratio in the range of 1.19 to 1.25, including each value within the specified range. In additional embodiments, the composition has a Hausner ratio in the range of 1.12 to 1.18, including each value within the specified range. In further embodiments, the composition has a Hausner ratio in the range of 1.00 to 1.11, including each value within the specified range.

In some embodiments, the carrier comprises at least one of a single type of cyclodextrin (modified or unmodified), a combination of different types of cyclodextrins (each, independently, modified or unmodified), lactose, starch, mannitol, microcrystalline cellulose, dextrin, maltodextrin, and a mixture or combination thereof. Each possibility represents a separate embodiment. In various embodiments, the carrier comprises a cyclodextrin selected from α-cyclodextrin, β-cyclodextrin, γ-cyclodextrin, 2-hydroxypropyl-β-cyclodextrin, methyl-β-cyclodextrin, and a mixture or combination thereof. Each possibility represents a separate embodiment. In further embodiments, the carrier comprises a sulfobutylether β-cyclodextrin. In other embodiments, the carrier comprises a sugar selected from sucrose, dextrose, molasses, lactose, and a mixture or combination thereof. Each possibility represents a separate embodiment. In specific embodiments, the carrier comprises lactose. In additional embodiments, the carrier comprises a sugar alcohol selected from mannitol, sorbitol, maltitol, xylitol, arabitol, isomalt, erythritol, glycerol, lactitol, and a mixture or combination thereof. Each possibility represents a separate embodiment. In particular embodiments, the carrier comprises mannitol. In yet other embodiments, the carrier comprises an inorganic mineral. In some embodiments, the carrier comprises silica, particularly mesoporous silica.

According to yet another aspect, there is provided a solid cannabinoid composition in the form of a powder, the composition comprising a carrier and a plurality of cannabinoids having a chemical signature which is substantially identical to the chemical signature of the plurality of cannabinoids in the extract of Cannabis, wherein the solid cannabinoid composition has a Hausner ratio of less than 1.59. In some embodiments, there is provided a solid cannabinoid composition in the form of a powder, the composition consisting essentially of a carrier and a plurality of cannabinoids having a chemical signature which is substantially identical to the chemical signature of the plurality of cannabinoids in the extract of Cannabis, wherein the solid cannabinoid composition has a Hausner ratio of less than 1.59. In other embodiments, there is provided a solid cannabinoid composition in the form of a powder, the composition consisting of a carrier and a plurality of cannabinoids having a chemical signature which is substantially identical to the chemical signature of the plurality of cannabinoids in the extract of Cannabis, wherein the solid cannabinoid composition has a Hausner ratio of less than 1.59.

In one embodiment, the weight percent ratio of the plurality of cannabinoids to the carrier in the composition is in the range of about 1:1 to about 1:20, including all iterations of ratios within the specified range. In another embodiment, the weight percent ratio of the plurality of cannabinoids to the carrier is in the range of about 1:5 to about 1:15, including all iterations of ratios within the specified range. In exemplary embodiments, the weight percent ratio of the plurality of cannabinoids to the carrier is about 1:10.

In particular embodiments, there is provided a solid cannabinoid composition consisting essentially of a cyclodextrin and a plurality of cannabinoids having a chemical signature which is substantially identical to the chemical signature of the plurality of cannabinoids in the extract of Cannabis, wherein the solid cannabinoid composition has a Hausner ratio of less than 1.59. In particular embodiments, there is provided a solid cannabinoid composition consisting of a cyclodextrin and a plurality of cannabinoids having a chemical signature which is substantially identical to the chemical signature of the plurality of cannabinoids in the extract of Cannabis, wherein the solid cannabinoid composition has a Hausner ratio of less than 1.59.

In other embodiments, there is provided a solid cannabinoid composition consisting essentially of lactose and a plurality of cannabinoids having a chemical signature which is substantially identical to the chemical signature of the plurality of cannabinoids in the extract of Cannabis, wherein the solid cannabinoid composition has a Hausner ratio of less than 1.59. In yet other embodiments, there is provided a solid cannabinoid composition consisting of lactose and a plurality of cannabinoids having a chemical signature which is substantially identical to the chemical signature of the plurality of cannabinoids in the extract of Cannabis, wherein the solid cannabinoid composition has a Hausner ratio of less than 1.59.

In some embodiments, there is provided a solid cannabinoid composition as disclosed herein which is a pharmaceutical composition for use in therapy. In other embodiments, there is provided a solid cannabinoid composition as disclosed herein for use in the treatment of a condition susceptible for treatment with a cannabinoid.

In certain embodiments, the composition is in a dosage form selected from tablet, pill, capsule, pellets, granules, powder, lozenge, sachet, cachet, elixir, suspension, dispersion, emulsion, solution, syrup, aerosol, gel, ointment, lotion, cream, and suppository, with each possibility representing a separate embodiment.

In various embodiments, the solid cannabinoid composition is adapted for administration via a route selected from oral, subcutaneous, intratracheal, intrabronchial, intra-alveolar, intraperitoneal, rectal, intravenous, intra-arterial, transdermal, intramuscular, topical, and intranasal, with each possibility representing a separate embodiment. In one embodiment, the solid cannabinoid composition is a powder adapted for use in a dry-powder inhaler. In another embodiment, the solid cannabinoid composition is a powder filled in a capsule for oral administration.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention.

In the Figures:

FIG. 1 schematically illustrates a method of preparing a solid cannabinoid composition according to one embodiment of the present invention.

FIG. 2 shows a photograph of inverted vials containing extracts of buds of a specific variety of Cannabis sativa.

FIG. 3A is an HPLC-UV trace of an ethanolic extract of buds of a specific variety of Cannabis sativa showing the cannabinoid profile of the variety.

FIG. 3B is an HPLC-UV trace of organic materials of a composition with a γ-cyclodextrin carrier according to the teachings herein.

FIG. 4A shows a photograph of a composition with a 2-hydroxypropyl-β-cyclodextrin carrier according to the teachings herein.

FIG. 4B shows a photograph of a vial containing a composition with a 2-hydroxypropyl-β-cyclodextrin carrier according to the teachings herein.

FIG. 5 is a heatmap showing the cannabinoid concentrations in a Cannabis extract vs. a composition with a methyl-β-cyclodextrin carrier (powder) according to the teachings herein.

FIG. 6 is an HPLC-UV trace of organic materials of a composition with a lactose carrier according to the teachings herein.

FIG. 7A shows the particle size distribution of a composition with a methyl-βcyclodextrin carrier subsequent to comminution in a vortex mill.

FIG. 7B shows the particle size distribution of a composition with a lactose carrier subsequent to comminution in a vortex mill.

FIG. 8 shows a scanning electron micrograph of a composition with a cyclodextrin carrier according to the teachings herein.

FIG. 9A is an HPLC-UV trace of a mixture of terpenes extract.

FIG. 9B is an HPLC-UV trace of terpene-containing powder according to the teachings herein.

FIG. 10 shows the in-vivo blood levels of CDB following administration of Cannabis extract (-♦-) and a composition according to the teachings herein (-▴-).

### DETAILED DESCRIPTION OF THE INVENTION

The present invention relates to solid compositions comprising cannabinoids as well as methods of making such solid compositions and uses thereof. Some embodiments relate to solid compositions comprising cannabinoids and other components extracted from Cannabis, e.g. terpenes. The compositions, in the form of a powder, are characterized by a chemical signature or fingerprint which mirrors the chemical signature of the extract of Cannabis from which it is obtained.

The principles, uses and implementations of the teachings of the invention may be better understood with reference to the accompanying description and figures. Upon perusal of the description and figures present herein, one skilled in the art is able to implement the teachings of the invention without undue effort or experimentation.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

In various fields, such as the fields of medicinal Cannabis and Cannabis commerce, it is important to be able to easily characterize the cannabinoid content of a given variety of Cannabis or of a product therefrom such as an extract. It is also important to obtain a Cannabis-based composition in which the different constituents of the plant work together in synergy to exert the entourage effect.

The inventors have found that it is possible to prepare a solid powder composition that comprises a carrier and a plurality of organic compounds extracted from Cannabis, in a simple and robust process. The composition is in the form of a solid powder that can be easily processed for use in a variety of applications. Unexpectedly, the composition can be prepared from an extremely viscous Cannabis extract even when the Cannabis extract is non-purified and contains other plant components such as chlorophyll, waxes and the like that are extracted from the Cannabis plant.

Also unexpectedly, it has been found that the cannabinoid fingerprint of the composition is a faithful representation of the cannabinoid fingerprint of the original extract from which the composition is prepared. Thus, the method provided herein is capable of preserving the synergy between different components of the Cannabis plant thereby preserving the entourage effect. The composition may be used as an active pharmaceutical ingredient containing a plurality of cannabinoids that act in synergy to afford high medical efficacy. The composition may also be useful as an adjuvant in pharmaceutical compositions comprising a non-cannabinoid active pharmaceutical ingredient. Additionally, the composition may also function as an easy-to-use surrogate for analysis, validation and evaluation of the original Cannabis extract.

In some embodiments, where the composition is produced by combining a non-purified Cannabis sativa extract with a suitable carrier, it is possible to release significant Cannabis sativa components other than cannabinoids from the composition for analysis.

Of particular importance is that the received powder is not only representative of the cannabinoids content of the sample from which it is made, but that it is easily used in an analytical laboratory setting for providing accurate and repeatable analytical results, inter alia, due to the powder being of uniform content and/or homogeneity so it can be easily weighed and otherwise manipulated.

According to some aspects and embodiments, provided herein is a method for preparing a solid powder composition comprising a plurality of organic compounds derived from a Cannabis extract. The extract of Cannabis is obtained from any species of the family Cannabaceae, for example Cannabis sativa, Cannabis indica, Cannabis ruderalis, and a mixture or combination thereof. Each possibility represents a separate embodiment. In one embodiment, the extract is obtained from a single Cannabis species or strain. The extract of Cannabis can also be obtained from hemp. It is to be understood that “hemp” as used herein refers to botanical hemp as well as to a Cannabis strain containing less than 0.3% THC. In one embodiment, the plurality of organic compounds comprises a plurality of cannabinoids. Within the plant, the major organ of production of cannabinoids is the inflorescence, particularly in epidermal hairs called glandular trichomes that are highly abundant on female inflorescences. In another embodiment, the plurality of organic compounds comprises a plurality of terpenes. In various embodiments, a Cannabis extract comprises a combination of cannabinoids extract and terpenes extract.

Suitable manners to obtain Cannabis extract within the scope of the present invention include, but are not limited to, organic solvent extraction, carbon dioxide (dry ice) extraction, supercritical and subcritical carbon dioxide extraction, hydrocarbon extraction, rosin press, and a combination thereof. Each possibility represents a separate embodiment. Typically, organic solvent extraction is performed using any of the following solvents and mixtures thereof including, but not limited to, ethanol, hexane, petroleum ether, methanol, chloroform and the like. Each possibility represents a separate embodiment. An extraction using a mixture of an organic solvent with water or an acid such as, but not limited to, acetic acid, formic acid, trifluoroacetic acid, and the like, are contemplated within the scope of the present invention. Carbon dioxide (dry ice) extraction, according to the principles of the present invention, can be followed by additional processing step(s) to provide an extract of Cannabis suitable for use in the method disclosed herein. Hydrocarbon extraction can be performed, for example using any gas suitable for extracting cannabinoids and other Cannabis components including, but not limited to, butane, propane, and the like. Each possibility represents a separate embodiment. It is to be understood that the aforementioned extractions can be performed at any temperature, for example below zero degrees centigrade, below room temperatures, at room temperatures, or at temperatures above room temperatures, with each possibility representing a separate embodiment. Currently preferred is the use of organic solvent extraction, particularly ethanolic extraction. In some embodiments, the extract is a crude extract that did not undergo a purification step. In other embodiments, the extract is a crude extract that was filtered. In yet other embodiments, the extract is a crude extract that was not filtered. In further embodiments, the extract is obtained following a purification step such as, but not limited to, wax or chlorophyll removal, winterization, distillation, and the like. Each possibility represents a separate embodiment. Suitable manners to obtain a terpene extract include, but are not limited to, distillation, steam distillation, hydrodistillation, supercritical and subcritical carbon dioxide extraction, hydrocarbon extraction, rosin, and organic solvent extraction. Each possibility represents a separate embodiment.

In some aspects and embodiments, the Cannabis extract, when dried to a residual solvent of about 5,000 ppm or less, is a non-powder crude oil having a viscosity of at least 3,000 cps as determined at 40° C. Residual amount of solvent of the Cannabis extract according to the principles of the present invention includes, but is not limited to, about 50 ppm to about 5,000 ppm, including each value within the specified range. For example, the amount of residual solvent in the Cannabis extract may be about 5,000 ppm, about 4,500 ppm, about 4,000 ppm, about 3,500 ppm, about 3,000 ppm, about 2,500 ppm, about 2,000 ppm, about 1,500 ppm, about 1,000 ppm, about 900 ppm, about 800 ppm, about 700 ppm, about 600 ppm, about 500 ppm, about 400 ppm, about 300 ppm, about 200 ppm, about 100 ppm, and about 50 ppm or less. Each possibility represents a separate embodiment. In certain embodiments, the Cannabis extract has about 5,000 ppm or less of residual solvent and a viscosity of at least 3,000 cps as determined at 40° C. It is contemplated that an extract can be obtained without the use of a solvent. In accordance with these embodiments, the extract is a non-powder crude oil having a viscosity of at least 3,000 cps as determined at 40° C.

Typical viscosities of a Cannabis extract crude oil, when dried to a residual solvent of about 5,000 ppm or less and measured at 40° C. include, but are not limited to, about 3,000 to about 2,000,000 cps, for example about 5,000 to about 1,000,000 cps, or about 10,000 to about 500,000 cps, including each value within the specified ranges. Viscosity can be measured as is known in the art using a suitable viscometer in a setup that is compatible for viscous materials. For example, viscosity can be measured using a viscometer such as, but not limited to, a Brookfield Viscometer or an Anton Paar Rheoplus viscometer with an appropriate setup. In one embodiment, viscosity of the extract can be measured using a Brookfield RTV viscometer with a LV-4 (64), a LV-5 (65), a RV/HA/HB-6 or a TE type spindle at 0.3-60 rpm. Each possibility represents a separate embodiment. In another embodiment, viscosity of the extract can be measured using a Brookfield DV-E viscometer with a LV-4 (64), a LV-5 (65), or a RV/HA/HB-6 type spindle at 0.3-60 rpm. Each possibility represents a separate embodiment. Exemplary non-limiting viscosities of a Cannabis extract when dried to a residual solvent of about 5,000 ppm or less and measured at 40° C., include about 3,000, about 4,000, about 5,000, about 6,000, about 7,000, about 8,000, about 9,000, about 10,000, about 15,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 95,000, about 100,000, about 125,000, about 150,000, about 175,000, about 200,000, about 225,000, about 250,000, about 275,000, about 300,000, about 325,000, about 350,000, about 375,000, about 400,000, about 425,000, about 450,000, about 475,000, about 500,000, about 525,000, about 550,000, about 575,000, about 600,000, about 625,000, about 650,000, about 675,000, about 700,000, about 725,000, about 750,000, about 775,000, about 800,000, about 825,000, about 850,000, about 875,000, about 900,000, about 925,000, about 950,000, about 975,000, about 1,000,000, about 1,100,000, about 1,200,000, about 1,300,000, about 1,400,000, about 1,500,000, about 1,600,000, about 1,700,000, about 1,800,000, about 1,900,000, or about 2,000,000 cps, with each possibility representing a separate embodiment.

According to certain aspects and embodiments, the extract is dissolved in a first solvent to afford a first solution. The first solvent is any suitable solvent that dissolves the provided extract. Suitable solvents within the scope of the present invention include, but are not limited to, C₁-C₈ aliphatic alcohols, C₁-C₁₀ aliphatic hydrocarbons, C₆-C₁₀ aromatic hydrocarbons, C₂-C₈ aliphatic esters, C₂-C₈ aliphatic ketones, C₄-C₈ ethers, C₁-C₁₀ halo-substituted aliphatic hydrocarbons, C₂-C₈ aliphatic amides, and a mixture or combination thereof. Each possibility represents a separate embodiment. Exemplary non-limiting organic solvents include ethanol, methanol, isopropyl alcohol, n-butanol, t-butyl alcohol, acetone, methyl ethyl ketone, toluene, benzene, hexane, cyclohexane, heptane, pentane, methyl acetate, ethyl acetate, t-butyl acetate, isopropyl acetate, diisopropyl ether, methyl t-butyl ether, tetrahydrofuran, dioxane, dichloromethane, dichloroethane, chloroform, carbon tetrachloride, tetrachloroethylene, tetrachloroethane, dimethylformamide, dimethylacetamide, and a mixture or combination thereof. Each possibility represents a separate embodiment. In currently preferred embodiments, the first solvent is miscible in water. In specific embodiments, the first solvent comprises not less than 70%, not less than 80%, not less than 90%, not less than 95% and even not less than 98% ethanol by weight. In one embodiment, the first solvent consists of a single type of solvent which is pure ethanol. In other embodiments, the first solvent consists of a combination of at least two different types of solvents, for example a combination of ethanol and ethyl acetate. In various embodiments, the step of dissolving the Cannabis extract in a first solvent to obtain a first solution is performed at room temperatures.

According to further aspects and embodiments, a carrier is then admixed with the first solution. In some embodiments, a dry carrier is added to the first solution. In other embodiments, the carrier is dissolved when added to the first solution. In alternative embodiments, the carrier does not dissolve when added to the first solution. When the carrier is not adequately soluble in the first solution, it is contemplated that the carrier may form a sediment and/or a suspension with the first solvent. In some embodiments, the required amount of carrier is added all at once to the first solution, preferably while mixing the first solution, e.g., by stirring or agitating, optionally while the temperature of the solution is elevated. However, it is to be understood that the step of adding a carrier to the first solution may be performed at room temperatures. In some embodiments, the required amount of carrier is added portion-wise or continuously, preferably while mixing the first solution, e.g., by stirring or agitating the first solution.

In further embodiments, the carrier is first dissolved or suspended in a second solvent and then admixed with the first solution to obtain a mixture comprising a plurality of cannabinoids and a carrier. In accordance with these embodiments, the first solution and the second solution or suspension can be prepared and mixed at any order. Typically, the second solution or suspension is added to the first solution while stirring or agitating the mixture as it is formed. The solvent used to dissolve or suspend the carrier is preferably miscible in the first solvent to afford a single solvent phase. Typically, an aqueous solvent is used to dissolve or suspend the carrier. Suitable solvents useful for dissolving or suspending the carrier within the scope of the present invention include, but are not limited to, water, acetone, ethanol, methanol, dimethyl formamide, DMSO, and a mixture or combination thereof. Each possibility represents a separate embodiment. In some preferred embodiments, the second solvent comprises not less than 70%, not less than 80%, not less than 90%, not less than 95% and even not less than 98% water by weight. In some embodiments, the second solvent consists of a single type of solvent such as pure water. In other embodiments, the second solvent consists of a combination of at least two different types of solvents, for example a combination of water and ethanol. In some embodiments, the two solutions or the solution and suspension are combined by adding one (portion-wise or continuously) into a vessel containing the other in small amounts with continuous mixing, e.g., by agitation or stirring, in a manner analogous to a titration. In other embodiments, the two solutions or the solution and suspension are combined by pouring both into a third vessel and mixing, e.g., by agitation or stirring.

The ratio between the first and second solvent is in the range of about 1:10 to about 10:1, for example from about 1:5 to about 5:1, and from about 1:5 to about 1:1, including all iterations of ratios within the specified ranges. Exemplary non-limiting ratios of the first to second solvent include about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1, with each possibility representing a separate embodiment. Currently preferred is a ratio of about 1:2.

According to various aspects and embodiments, removal of the solvent(s) is performed using at least one of the following techniques: evaporation, optionally at elevated temperatures and/or reduced pressures, freeze-drying (lyophilization), distillation, air drying, spray drying, fluid bed drying, or a combination thereof. Each possibility represents a separate embodiment. In currently preferred embodiments, removal of the solvent(s) is performed using a combination of evaporation and lyophilization. It is contemplated that removal of solvent(s) can be performed in a plurality of sequential steps, wherein each step comprises removal of a portion of the solvent(s). In accordance with these embodiments, the technique used for removing a portion of the solvent in one step can be the same or different than the technique used for removing a portion of the solvent in another step, with each possibility representing a separate embodiment. In some embodiments, solvent removal is performed at elevated temperatures. Typically, temperatures in the range of about 30° C. to about 70° C. can be used, including each value within the specified range.

In some embodiments, removing the solvent(s) is such that at least 90% by weight of the solvent(s) is removed from the mixture. In other embodiments, at least 95% by weight of the solvent(s) is removed from the mixture. In yet other embodiments, even at least 97% by weight of the solvent(s) is removed from the mixture. It is contemplated that upon removal of at least 90% by weight of the solvent(s), the obtained composition has a Loss on Drying (LOD) % of 10% or less, for example 0.01% to 10%, including each value within the specified range. In certain embodiments, the obtained composition has a LOD % of 5% or less, for example 0.01% to 5%, including each value within the specified range. According to the principles of the present invention, following solvent removal, a solid residue which is the solid powder composition disclosed herein is provided. In some embodiments, following solvent removal, a solid residue is provided, which can be further processed, for example by grinding, to yield the solid powder composition of the present invention.

The method according to the principles of the present invention may further comprise additional processing steps including, but not limited to, comminution, sieving, heating, drying, lubricating, and packaging as is known in the art. Each possibility represents a separate embodiment. In some embodiments of the method, the average particle size of the solid powder composition is further reduced, for example by comminution. Alternatively or additionally, in some embodiments of the method, the average particle size of the solid powder composition undergoes size separation to produce at least two portions of different average particle sizes. Comminution can be performed using any suitable method, e.g., milling, grinding, crushing, cutting, using any suitable device, e.g., vortex mill, jet mill, conical mill, ball mill, SAG mill, pebble mill, roller press, buhrstone mill, VSI mill, tower mill or combinations thereof. Each possibility represents a separate embodiment. Additional processing steps that are useful for implementing the teachings are well-known to a person having ordinary skill in the art and may include such methods as conventional mixing, dissolving, emulsifying etc. Each possibility represents a separate embodiment.

In some embodiments, comminution is performed to reduce the average particle size at least by half. In other embodiments, comminution is performed to reduce the average particle size to not greater than 500 micrometers, not greater than 250 micrometers, not greater than 100 micrometers, not greater than 50 micrometers, and even not greater than 25 micrometers. Each possibility represents a separate embodiment.

Size separation can be performed using any suitable method, e.g., mechanical sieving, cyclonic separation, etc. In some embodiments, size separation is performed to yield a portion having an average particle size of not greater than 500 micrometers, not greater than 250 micrometers, not greater than 100 micrometers, not greater than 50 micrometers, not greater than 25 micrometers, not greater than 10 micrometers, and even not greater than 5 micrometers. Each possibility represents a separate embodiment. In certain embodiments, size separation is performed to eliminate oversized agglomerates.

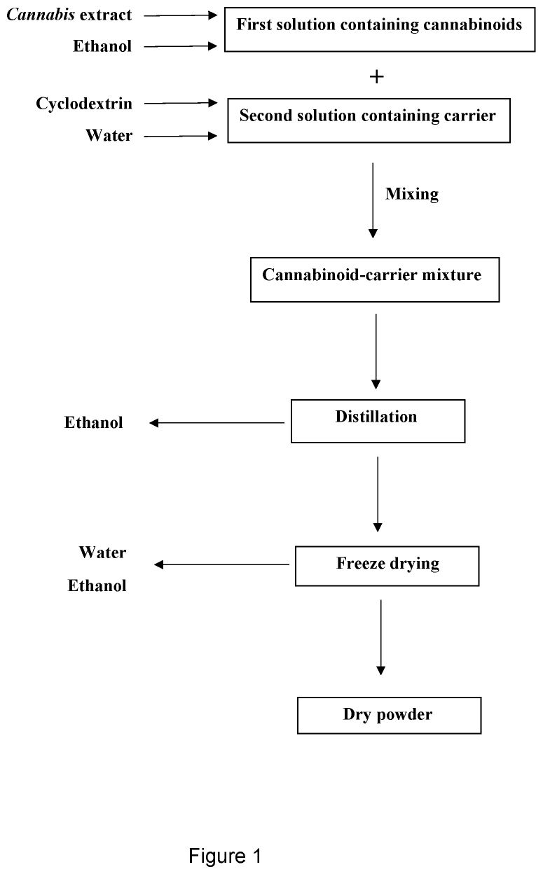

Referring now to the drawings, FIG. 1 illustrates a flowchart of a method of preparing a solid cannabinoid composition according to one embodiment of the present invention. In particular, an extract of Cannabis is dissolved in ethanol to obtain a first solution comprising a plurality of cannabinoids. A second solution is obtained by dissolving a water-soluble cyclodextrin in water. The second solution is gradually added to the first solution followed by partial removal of the ethanol by distillation. Residual ethanol and water are then removed using freeze drying (lyophilization).

According to various aspects and embodiments of the present invention, there is provided a solid composition, comprising: a carrier; and a plurality of organic compounds extracted from Cannabis, wherein the plurality of organic compounds includes cannabinoids. The plurality of organic compounds extracted from Cannabis are physically associated with the carrier. The composition, according to the principles provided herein is a powder, preferably a flowable and dispersible powder. In one embodiment, the powder is water soluble. In another embodiment, the powder is water insoluble. In some embodiments, the composition is prepared by the method disclosed herein.

According to the principles provided herein, the composition comprises a plurality of cannabinoids having a chemical signature which is substantially identical to the chemical signature of the plurality of cannabinoids in the extract of Cannabis from which it is derived. A chemical signature of the plurality of cannabinoids can be determined by any analytical method known in the art including, but not limited to, gas chromatography (GC), high pressure liquid chromatography (HPLC), thin layer chromatography (TLC), and infra-red (IR) spectrometry. Each possibility represents a separate embodiment. The aforementioned techniques can be used with different detectors such as, but not limited to, UV/PDA, mass spectrometry (MS), and flame ionization detector (FID). Each possibility represents a separate embodiment. As used herein and in the appended claims, the term “a chemical signature which is substantially identical to the chemical signature of the plurality of cannabinoids in the extract of Cannabis” refers to a profile of the composition of the present invention as determined by any of the methods disclosed herein which contains features (e.g. peaks) that are at least 50%, at least 60%, at least 70%, at least 80%, at least 90%, or even at least 95% identical in their appearance and relative intensities as compared to the features detected from the extract of Cannabis when using the same detection technique and parameters. Each possibility represents a separate embodiment. For example, when using HPLC, “a chemical signature which is substantially identical to the chemical signature of the plurality of cannabinoids in the extract of Cannabis” refers to a profile which contains peaks that are at least 50%, at least 60%, at least 70%, at least 80%, at least 90%, or even at least 95% identical in their retention time or relative distance from the starting point as well as the relative peak intensities as compared to the peaks detected from the extract of Cannabis when using the same measurement parameters. It is to be understood that following a measurement, the data can be further processed to yield a pattern (for example a heatmap). In accordance with these embodiments, the term “a chemical signature which is substantially identical to the chemical signature of the plurality of cannabinoids in the extract of Cannabis” refers to a pattern which is at least 50%, at least 60%, at least 70%, at least 80%, at least 90%, or even at least 95% identical in its appearance as compared to the pattern detected from the extract of Cannabis, when using the same processing algorithm.

In certain embodiments, the composition comprises two or more cannabinoids selected from the group consisting of cannabidivarinic acid (CBDVA), cannabidiolic acid (CBDA), cannabigerolic acid (CBGA), cannabidiol (CBD), cannabinol (CBN), cannabinolic acid (CBNA), tetrahydrocannabinol (THC), cannabichromene (CBC), cannabichromenic acid (CBCA), tetrahydrocannabinolic acid (THCA), cannabicitran, and a mixture or combination thereof. Each possibility represents a separate embodiment. In one embodiment, the composition comprises two of the aforementioned cannabinoids. In another embodiment, the composition comprises three of the aforementioned cannabinoids. In yet another embodiment, the composition comprises four of the aforementioned cannabinoids. In additional embodiments, the composition comprises five of the aforementioned cannabinoids. In further embodiments, the composition comprises six of the aforementioned cannabinoids. In other embodiments, the composition comprises seven of the aforementioned cannabinoids. In certain embodiments, the composition comprises eight of the aforementioned cannabinoids. In other embodiments, the composition comprises nine of the aforementioned cannabinoids. In yet other embodiment, the composition comprises ten of the aforementioned cannabinoids. In particular embodiments, the composition comprises all of the aforementioned cannabinoids. Additional cannabinoids that may be included in the compositions of the present invention are phytocannabinoids identified and characterized according to Berman et al. (2018, Sci. Rep. 8(1):14280-14294) such as, but not limited to, tetrahydrocannabivarin (THCV), cannabigerol (CBG), sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabigerolic acid monomethyl ether (CBGMA), cannabigerol monomethyl ether (CBGM), cannabicyclol (CBL), cannabicyclolic acid (CBLA), THCA-C4, THC-C4, tetrahydrocannabivarin carboxylic acid (THCVA), tetrahydrocannabivarin (THCV), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol (CBN), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, and a mixture or combination thereof. Each possibility represents a separate embodiment. In some embodiments, the composition further comprises at least one non-cannabinoid phytochemical extracted from a plant in the genus Cannabis. In accordance with these embodiments, other components extracted from a plant in the genus Cannabis may also be included in the composition. For example, the composition may further include at least one of terpenes, terpenoids, flavonoids, nitrogenous compounds, amino acids, proteins, glycoproteins, sugars, hydrocarbons, fatty acids, esters, lactones, steroids, non-cannabinoid phenols, and a mixture or combination thereof. Each possibility represents a separate embodiment. In some embodiments, a plurality of terpenes, terpenoids, flavonoids, or combinations thereof extracted from Cannabis is further added to the Cannabis extract and subjected to the process steps disclosed herein to afford a solid cannabinoid composition comprising a plurality of cannabinoids and further comprising a plurality of terpenes, terpenoids, flavonoids, or combinations thereof. Each possibility represents a separate embodiment. In various embodiments, a plurality of terpenes which may be naturally occurring (for example from a Cannabis plant or another plant source, flowers, fruits, etc.), synthetic or semi-synthetic is added to the Cannabis extract. In other embodiments, the composition disclosed herein is devoid of terpenes.

According to some aspects and embodiments, the solid cannabinoid composition is a flowable powder. In some embodiments, flowable means that the composition can be milled and/or sieved as a step in size-separation of the constituent particles of the composition. In various embodiments, particles of greater than 300 micrometers in size can be separated from particles smaller than 300 micrometers by sieving through an agitated stainless steel 300 micrometer mesh sieve. In other embodiments, the term “flowable” as used herein refers to a composition having a Hausner ratio of less than 1.59, for example between 1.00 and 1.59, including each value within the specified range. Suitable, non-limiting, Hausner ratios within the scope of the present invention include ranges of 1.46 to 1.59, 1.35 to 1.45, 1.26 to 1.34, 1.19 to 1.25, 1.12 to 1.18, and 1.00 to 1.11, including each value within the specified ranges. Hausner ratio, as used herein, can be calculated according to the following equation: H=(ρ tapped)/(ρ bulk), where “H” designates the Hausner ratio, “ρ tapped” designates the tapped density, and “ρ bulk” designates the bulk density.

Some embodiments of a composition according to the teachings herein are comminutable, that is to say, the average particle size can be reduced by grinding, for example using a mortar and pestle without concomitant release of the organic compounds as a fluid such as an oil. Without being bound by any theory or mechanism of action, it is contemplated that the lack of oil release is attributed to the unexpected association between carrier molecules and the plurality of cannabinoids and/or other Cannabis components.

The average particle size and distribution of a composition according to the teachings herein is any suitable average particle size and distribution. In some embodiments, the particle size is similar or substantially identical to the particle size of the carrier used, although in some embodiments the average particle size and/or distribution is different. Each possibility represents a separate embodiment.

Typically, the average particle size of the solid cannabinoid composition of the present invention is between 10 nanometers and 2 mm, including each value within the specified range. In some embodiments, the average particle size is not less than 100 nanometers, not less than 500 nanometers, and even not less than 1 micrometer, with each possibility representing a separate embodiment. In other embodiments, the average particle size is not greater than 1 mm, not greater than 500 micrometers, not greater than 100 micrometers, not greater than 50 micrometers, and even not greater than 10 micrometers, with each possibility representing a separate embodiment.

According to some aspects and embodiments, the composition according to the teachings herein comprises a plurality of organic compounds extracted from Cannabis which are associated with a carrier to form a powder composition. In one embodiment, the composition comprises a plurality of cannabinoids associated with the carrier. According to other aspects and embodiments, the composition according to the teachings herein consists essentially of a carrier and a plurality of cannabinoids. According to further aspects and embodiments, the composition according to the teachings herein consists of a carrier and a plurality of cannabinoids. Typically, the carrier is a solid carrier at standard temperature and pressure (STP). In one embodiment, the carrier is a pharmaceutically acceptable carrier. In several embodiments, the carrier is a pharmaceutical grade carrier. In some embodiments, a composition comprising a single type of carrier is provided. In other embodiments, a composition comprising at least two different types of carrier is provided. In certain embodiments, the weight percent ratio of the plurality of cannabinoids to the carrier in the composition is in the range of about 1:1 to about 1:20, for example from about 1:5 to about 1:15, including all iterations of ratios within the specified ranges. Exemplary, non-limiting, ratios of the plurality of cannabinoids to the carrier include about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20, with each possibility representing a separate embodiment. In currently preferred embodiments, the weight percent ratio of the plurality of cannabinoids to the carrier is about 1:10.

In some embodiments, the carrier comprises at least one cyclodextrin selected from the group consisting of a single type of cyclodextrin (modified or unmodified), a combination of different types of cyclodextrins (each, independently, modified or unmodified), lactose, starch, mannitol, microcrystalline cellulose, dextrin, maltodextrin, and a mixture or combination thereof. Each possibility represents a separate embodiment. In one embodiment, the composition is devoid of triglycerides or fatty acids. In other embodiments, the composition is devoid of phospholipids. In further embodiments, the composition is devoid of caffeine. In various embodiments, the composition is devoid of emulsifying agents. In yet other embodiments, the composition is devoid of surfactants.

In certain embodiments, the carrier comprises a cyclodextrin selected from the group consisting of a single type of cyclodextrin and a combination of at least two different types of cyclodextrins. Each possibility represents a separate embodiment. In some embodiments, the carrier comprises not less than 50%, not less than 60%, not less than 70%, not less than 80%, not less than 90%, and even not less than 95% by weight cyclodextrin(s). In certain embodiments, the carrier consists essentially of cyclodextrin(s). In various embodiments, the carrier consists of cyclodextrin(s). Suitable types of cyclodextrins include, but are not limited to, α-cyclodextrin, β-cyclodextrin, γ-cyclodextrin, 2-hydroxypropyl-β-cyclodextrin, methyl-β-cyclodextrin, and a mixture or combination thereof. Each possibility represents a separate embodiment. Additional cyclodextrins within the scope of the present invention include, but are not limited to, heptakis (2,3,6-tri-O-methyl)-β-cyclodextrin, heptakis (2,6-di-O-methyl)-β-cyclodextrin, sulfopropylated-heptakis (2,6-di-O-methyl)-β-cyclodextrin, carboxymethylated-heptakis (2,6-di-O-methyl)-β-cyclodextrin sodium, carboxymethyl-hydroxyethylated-heptakis (2,6-di-O-methyl)-β-cyclodextrin, succinylated methyl-β-cyclodextrin, methylated-6-monodeoxy-6-monoamino-β-cyclodextrin, quaternary amino β-cyclodextrin, and carboxymethylated-β-cyclodextrin. Each possibility represents a separate embodiment. In one embodiment, the cyclodextrin is a modified cyclodextrin. Currently preferred is the use of 2-hydroxypropyl-β-cyclodextrin as the carrier. Unexpectedly, 2-hydroxypropyl-β-cyclodextrin provides a water-soluble powder composition which is particularly suitable for oral administration. An additional suitable cyclodextrin within the scope of the present invention includes a sulfobutylether β-cyclodextrin. In one embodiment, the composition is devoid of sulfoalkylether cyclodextrin. Additional commercially available cyclodextrin within the scope of the present invention is Captisol (SBE-β-cyclodextrin).

In some embodiments, the carrier comprises a sugar selected from sucrose, dextrose, molasses, lactose, and a mixture or combination thereof. Each possibility represents a separate embodiment. In particular embodiments, the carrier is lactose, preferably D-(+)-lactose, more preferably D-(+)-lactose monohydrate. In various embodiments, the carrier comprises not less than 50%, not less than 60%, not less than 70%, not less than 80%, not less than 90%, and even not less than 95% by weight lactose. In some embodiments, the carrier consists essentially of lactose. As lactose is considered inert without causing adverse local effects to the lungs, it is contemplated that the use of lactose as a carrier provides a powder composition which is particularly suitable for administration via inhalation. In certain embodiments, the carrier consists of lactose. In one embodiment, the composition is devoid of maltodextrin.

In additional embodiments, the carrier comprises a sugar alcohol. Suitable sugar alcohols within the scope of the present invention include, but are not limited to, mannitol, sorbitol, maltitol, xylitol, arabitol, isomalt, erythritol, glycerol, lactitol, and a mixture or combination thereof. Each possibility represents a separate embodiment. In particular embodiments, the carrier comprises mannitol. In some embodiments, the carrier comprises not less than 50%, not less than 60%, not less than 70%, not less than 80%, not less than 90%, and even not less than 95% by weight mannitol. In certain embodiments, the carrier consists essentially of mannitol. In various embodiments, the carrier consists of mannitol.

In further embodiments, the carrier comprises an inorganic mineral. Suitable inorganic minerals include, but are not limited to, silica and mesoporous silica. Each possibility represents a separate embodiment. In particular embodiments, the carrier comprises mesoporous silica. In some embodiments, the carrier comprises not less than 50%, not less than 60%, not less than 70%, not less than 80%, not less than 90%, and even not less than 95% by weight mesoporous silica. In certain embodiments, the carrier consists essentially of mesoporous silica. In various embodiments, the carrier consists of mesoporous silica.

The solid cannabinoid composition according to the principles of the present invention can be administered as a pharmaceutical composition. In accordance with these embodiments, the carrier is a pharmaceutically acceptable carrier. In some embodiments, the plurality of cannabinoids associated with the carrier is used as an active pharmaceutical ingredient. In various embodiments, there is provided a solid cannabinoid composition as disclosed herein for use in therapy, for example in treating a condition susceptible for treatment with a cannabinoid. The term “treating” as used herein refers to stopping or slowing down the progression of a disease. This term also includes the reduction in the occurrence of various symptoms associated with a disease. Surprisingly, it has been found that the composition of the present invention exerts improved bioavailability as compared to the Cannabis extract from which it is obtained. It is contemplated that at least some of the cannabinoids and/or other Cannabis components are bioavailable such that the synergy between different Cannabis components is maintained. Accordingly, the compositions disclosed herein provide increased medical efficacy. The compositions containing a therapeutically effective amount of cannabinoids and/or other Cannabis components can therefore be administered to a human or non-human mammal, preferably a human, in an easy-to-use dosage form with improved patient compliance. Suitable dosage forms within the scope of the present invention include, but are not limited to, tablet, pill, capsule, pellets, granules, powder, lozenge, sachet, cachet, elixir, suspension, dispersion, emulsion, solution, syrup, aerosol, gel, ointment, lotion, cream, and suppository. Each possibility represents a separate embodiment.

In some embodiments, the pharmaceutical composition is in the form of a powder. In certain embodiments, the pharmaceutical composition is a powder for use in a dry-powder inhaler. In accordance with these embodiments, the size distribution of the particles constituting the powder is suitable for administration using a dry-powder inhaler as described, for example in U.S. Pat. No. 9,056,173. Within the scope of the present invention is a kit for medical treatment comprising:

a dry-powder inhaler; and

a composition according to the teachings herein,

wherein the composition is charged to the dry-powder inhaler to afford administration of the composition to the lungs of a subject in need thereof. In such embodiments, the particle size of the powder is a particle size suitable for administration as a dry powder. For administration via inhalation, typically the particle size of the cannabinoid-bearing powder is in the range of about 0.01 to about 15 micrometers, including each value within the specified range. Currently preferred average particle size is in the range of about 0.1 to about 15 micrometers, including each value within the specified range. According to other embodiments, the particle size is in the range of about 1 to about 5 micrometers, including each value within the specified range.

In various embodiments, the pharmaceutical composition is a fluid and further comprises a fluid matrix. In other embodiments, the powder is dissolved or suspended in the fluid matrix to form a pharmaceutical composition in the form of a solution or suspension. In yet other embodiments, the solution or suspension is encapsulated in a solid capsule, e.g., a gel-cap. In further embodiments, the solution or suspension is formulated as eye drops. In one embodiment, the solution or suspension is not a liposomal composition.

In certain embodiments, the pharmaceutical composition is a gel and further comprises a gel matrix. In other embodiments, the powder is suspended in the gel matrix. In additional embodiments, the composition is formulated as a cream or a lotion.

In some embodiments, the pharmaceutical composition is a solid mass (such as a tablet). In other embodiments, the powder is present in the pharmaceutical composition as a sintered powder (e.g., sintered by heat and/or pressure). In additional embodiments, the composition is encapsulated in a solid capsule, e.g., a hard-shell capsule.

In various embodiments, the pharmaceutical composition further comprises at least one excipient. Suitable excipients include, but are not limited to, a binder, a filler, a bulking agent, a surfactant, an anti-tacking agent, a plasticizer, a lubricant, a glidant, a disintegrant, a diluent, a tonicity enhancing agent, a wetting agent, a buffering substance, a colorant, a preservative, and any combination thereof, with each possibility representing a separate embodiment.

Suitable routes of administration include, but are not limited to, oral, subcutaneous, intratracheal, intrabronchial, intra-alveolar, intraperitoneal, rectal, intravenous, intra-arterial, transdermal, intramuscular, topical, and intranasal. Each possibility represents a separate embodiment. According to certain embodiments, the compositions are suitable for oral administration. It is contemplated that by orally administering the compositions, a systemic effect can be achieved.

In one embodiment, the compositions are administered through the nasal respiratory route. Compositions in pharmaceutically acceptable solvents may be nebulized by use of inert gases. Nebulized solutions may be breathed directly from the nebulizing device or the nebulizing device may be attached to a face mask tent, or intermittent positive pressure breathing machine. Solution, suspension, or powder compositions may be administered, orally or nasally, from devices that deliver the composition in an appropriate manner.

The administration regimen can be determined by a skilled artisan depending on various parameters including the patient population, age, weight etc. The amount of the cannabinoids to be administered in order to confer effective treatment depends on the nature of the disorder or condition to be treated, and can be determined by clinical techniques. In addition, in vitro assays, in vivo assays and ex-vivo assays may optionally be employed to help identify optimal dose ranges. The precise dose to be employed also depends on the route of administration, and the progression of the disease or disorder, and should be decided according to the judgment of the practitioner and each patient's circumstances. Typically, doses in the range of 0.001 to 1,000 mg/kg of body weight, 0.01 mg/kg to 100 mg/kg, 0.1 mg/kg to 100 mg/kg, 1 mg/kg to 100 mg/kg, 10 mg/kg to 75 mg/kg, etc. may be used. Each possibility represents a separate embodiment. Exemplary, non-limiting amounts include 0.5 mg/kg, 1 mg/kg, 5 mg/kg, 10 mg/kg, 20 mg/kg, 50 mg/kg, 60 mg/kg, 75 mg/kg, and 100 mg/kg, with each possibility representing a separate embodiment. Effective doses may be extrapolated from dose-response curves derived from in vitro, animal model or ex-vivo model test bioassays or systems. Typical fixed doses include, but not limited to, 5 mg, 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1,000 mg, with each possibility representing a separate embodiment.

The administration schedule can be taken once-daily, twice-daily, thrice-daily, once-weekly, twice-weekly, thrice-weekly, once-monthly, twice-monthly, thrice-monthly, or any other administration schedule known to those of skill in the art. Provided herein are pharmaceutical compositions that exhibit release profiles that comprise all possible modes of release profiles including, but not limited to, immediate release (IR), or modified release such as delayed release (DR), sustained release (SR) and extended release (XR) formulations. Each possibility represents a separate embodiment. In currently preferred embodiments, the release profile is immediate release and the composition is devoid of any sustained release polymers/agents. In addition, the administration can be continuous, i.e., every day, or intermittent. The terms “intermittent” or “intermittently” as used herein refer to stopping and starting at either regular or irregular intervals. For example, intermittent administration can be administration in one to six days per week or it may mean administration in cycles (e.g. daily administration for two to eight consecutive weeks, then a rest period with no administration for up to one week) or it may mean administration on alternate days. In additional embodiments, “intermittent” or “intermittently” refers to a sporadic use.

Within the scope of the present invention is the treatment of neuropathic pain, cancer and adverse effects caused by chemotherapeutic agents, Parkinson's disease, Alzheimer's disease, Autism, fibromyalgia, post-traumatic stress disorder, Amyotrophic Lateral Sclerosis (ALS), epilepsy, AIDS, ulcerative colitis, Crohn's disease, rheumatoid arthritis, Tourette syndrome, multiple sclerosis, opiate or opioid addiction withdrawal, and diabetes. Each possibility represents a separate embodiment. Additional disorders that can be treated with the composition of the present invention include, but are not limited to, small airway disease, chronic bronchitis, emphysema, chronic obstructive pulmonary disease (COPD), cystic fibrosis, bronchiectasis, asthma, pneumonia, parenchymatic and fibrotic lung diseases or disorders, interstitial pulmonary fibrosis, and sarcoidosis. Each possibility represents a separate embodiment. In accordance with these embodiments, the cannabinoid composition disclosed herein is useful as monotherapy.

In certain aspects and embodiment, the composition comprising a plurality of cannabinoids according to the present invention may be used as an adjuvant in a dosage form comprising non-cannabinoid active pharmaceutical ingredients thereby facilitating their use. In certain embodiments, the non-cannabinoid active pharmaceutical ingredient is not a phytochemical. In other embodiments, a composition comprising a non-cannabinoid active pharmaceutical ingredient may be administered as separate dosage form for combined administration with the cannabinoid composition of the present invention. It is contemplated that the cannabinoid composition of the present invention is suitable as an add-on therapy as well as a complementary therapy to non-cannabinoid drug therapy. In certain embodiments, the composition disclosed herein may be used to reduce the occurrence of any adverse events caused by the co-administration with a non-cannabinoid active pharmaceutical ingredient. Combined administration in the context of this invention is defined to mean the administration of both compositions in the course of a coordinated treatment to achieve an improved clinical outcome. Such combined administration may occur at the same time and also be coextensive, that is, occurring during overlapping periods of time. Combined administration, as used herein, refers to a regimen selected from: a single combined composition, separate individual compositions administered substantially at the same time, and separate individual compositions administered under separate schedules. Each possibility represents a separate embodiment.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the use of “a” and “an” means “at least one” or “one or more” unless the context clearly dictates otherwise.

As used herein, when a numerical value is preceded by the term “about”, the term “about” is intended to indicate ±10%. As used herein, room temperatures refer to 25±5° C.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

### Materials

Unless otherwise noted, materials were purchased from commercial sources such as Sigma-Aldrich Israel Ltd. (Rehovot, Israel) and Bio-lab Ltd. (Jerusalem, Israel). Cyclodextrins were purchased from Glentham Life Sciences (Corsham, UK).

### HPLC Methods

HPLC-UV analyses of cannabinoid- and terpene-based extracts and the compositions disclosed herein were performed as follows:

Column: Halo® C18, 2.1×150, 2.7 micrometer (Part No. 92812-702 by Advanced Materials Technology, Wilmington, Del., USA).

Eluents: Line A: 0.1% TFA in water; Line B: 0.1% TFA in acetonitrile; Line C: methanol.

Conditions: PDA from 190-400, channel 1: 220 nm; channel 2: 263 nm; channel 3: 207 nm; and channel 4: 274 nm.

Column temperature: 300° C.±10° C.

Autosampler temperature: 40° C.±10° C.

Flow rate: 0.25 mL/min.

Injection volume: 1 μL.

TABLE 1
Gradient:

| Time (mm) | A (%) | B (%) | C (%) |
|---|---|---|---|
| 0 | 45 | 50 | 5 |
| 3 | 30 | 67 | 3 |
| 8 | 30 | 67 | 3 |
| 11 | 21 | 74 | 5 |
| 14 | 21 | 74 | 5 |
| 18 | 5 | 90 | 5 |
| 21 | 5 | 90 | 5 |
| 21.1 | 45 | 50 | 5 |
| 27 | 45 | 50 | 5 |

### Preparation of an Embodiment of a Solid Cannabinoid Composition in the Form of a Powder

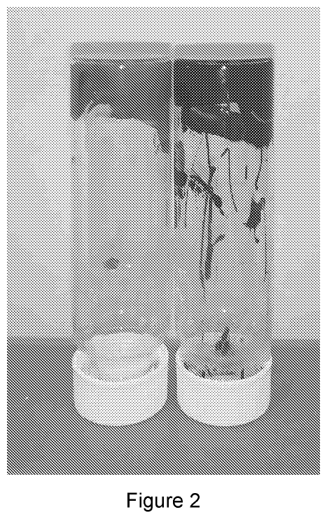

An ethanolic extract of Cannabis sativa buds with residual ethanol of less than 5,000 ppm (0.5%) was obtained as an extremely viscous and sticky brown tar in a round-bottomed flask. FIG. 2 shows a photograph of inverted vials containing an ethanolic extract (right) and a super critical carbon dioxide extract (left) of Cannabis sativa demonstrating the highly viscous crude oil consistency of the extracts.

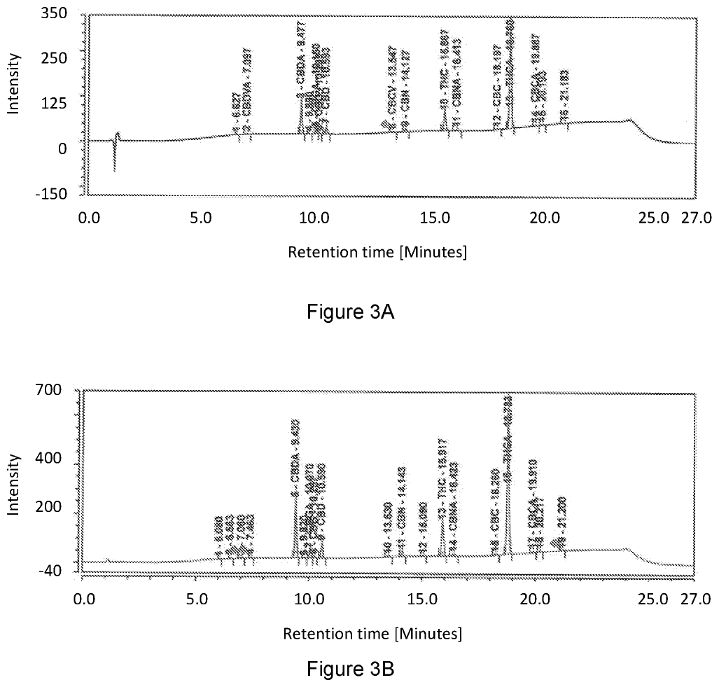

A sample of the ethanolic extract was dissolved in 100% ethanol and analyzed using HPLC-UV. The resulting HPLC-UV trace showed the cannabinoid fingerprint of the specific Cannabis sativa variety which was extracted (FIG. 3A).

A. Composition with γ-Cyclodextrin Carrier

0.347 gram of the Cannabis extract was dissolved in 12 ml 100% ethanol as a first liquid solvent at room temperature in a round-bottomed flask yielding a first cannabinoid solution. 3.12 gram of γ-cyclodextrin was dissolved in 13 ml water as a second liquid solvent yielding a second cyclodextrin solution. The cyclodextrin solution was added dropwise to the cannabinoid solution with vigorous stirring using a magnetic stirrer at room temperature. Ethanol was removed under reduced pressure (water aspirator) at 40° C. using a rotary evaporator followed by water removal using freeze-drying. After freeze-drying, a flowable light-yellow powder was obtained.

A sample of the powder was placed in a small amount of the mobile phase described above to release cannabinoid elements into the liquid. A sample of the liquid was analyzed using HPLC-UV as described above. The HPLC-UV trace obtained from the powder (FIG. 3B) showed the cannabinoid fingerprint of the specific Cannabis sativa variety which was used. The cannabinoids profile was substantially identical to the cannabinoid fingerprint of the untreated extract (FIG. 3A).

The powder was examined under a microscope and found to be heterogeneous in terms of size with particles ranging from 2 to 300 micrometers in diameter (largest dimension). In addition, a large variety of shapes was observed including flakes and grains.

A sample of the powder was manually milled using a mortar-and-pestle, and the resulting powder was observed under a microscope. The average particle size was reduced and no oil or other residues were observed. A sample of the powder was passed through a stainless steel 300 micrometer mesh sieve and flowed easily through the sieve.

B. Compositions with 2-Hydroxypropyl-β-Cyclodextrin Carrier

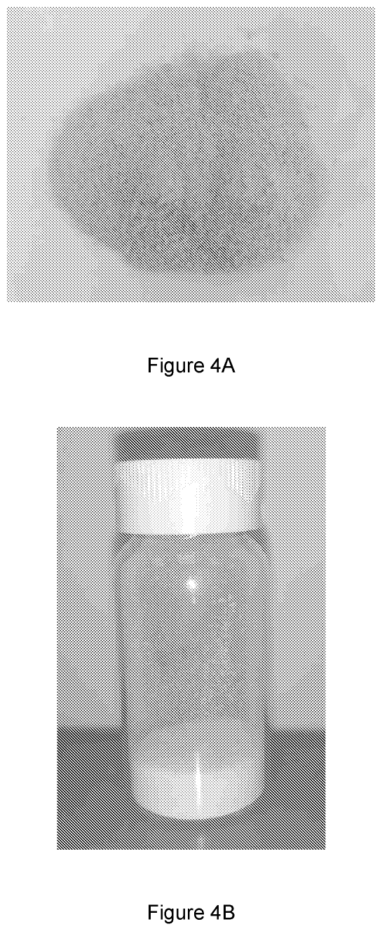

0.180 gram of the Cannabis extract was dissolved in 2 ml 100% ethanol as a first liquid solvent at room temperature in a round-bottomed flask yielding a first cannabinoid solution. 1.08 gram of 2-hydroxypropyl-β-cyclodextrin was dissolved in 3 ml water as a second liquid solvent yielding a second cyclodextrin solution. The cannabinoid solution was added to the cyclodextrin solution with vigorous stirring using a magnetic stirrer at room temperature. Subsequently, the solvents were removed from the flask under reduced pressure. 5 ml of solvent was added to the obtained mixture to obtain a slurry. Solvents were removed by filtration to obtain flowable light-yellow powder. FIGS. 4A and 4B show photographs of the obtained powder.

A composition prepared from Cannabis extract and 2-hydroxypropyl-β-cyclodextrin at a ratio of 1:9 was obtained according to the method of the present invention. The composition was milled to provide particles of less than 1,000 microns in size with a median particle size (d50) of 125 microns. The bulk and tapped densities of the milled composition were measured and the obtained Hausner ratio was 1.38.

C. Compositions with Methyl-β-Cyclodextrin Carrier

0.5 gram of the Cannabis extract was dissolved in 5 ml 100% ethanol as a first liquid solvent at room temperature in a round-bottomed flask yielding a first cannabinoid solution. 0.5 gram of methyl β-cyclodextrin was dissolved in 5 ml water as a second liquid solvent yielding a second cyclodextrin solution. The cyclodextrin solution was added dropwise to the cannabinoid solution with vigorous stirring using a magnetic stirrer at room temperature. Ethanol was removed from the flask under reduced pressure (water aspirator) at 40° C. using a rotary evaporator followed by water removal using freeze-drying. After freeze-drying, a flowable light-yellow powder remained in the flask.

4.4 gram of the Cannabis extract was dissolved in 150 ml 100% ethanol as a first liquid solvent at room temperature in a round-bottomed flask yielding a first cannabinoid solution. 43.2 gram of methyl-β-cyclodextrin was dissolved in 75 ml water as a second liquid solvent yielding a second cyclodextrin solution. The cyclodextrin solution was added dropwise to the cannabinoid solution with vigorous stirring using a magnetic stirrer at room temperature. Ethanol was removed from the flask under reduced pressure (water aspirator) at 40° C. using a rotary evaporator followed by water removal using freeze-drying. After freeze-drying, a flowable light-yellow powder remained in the flask.

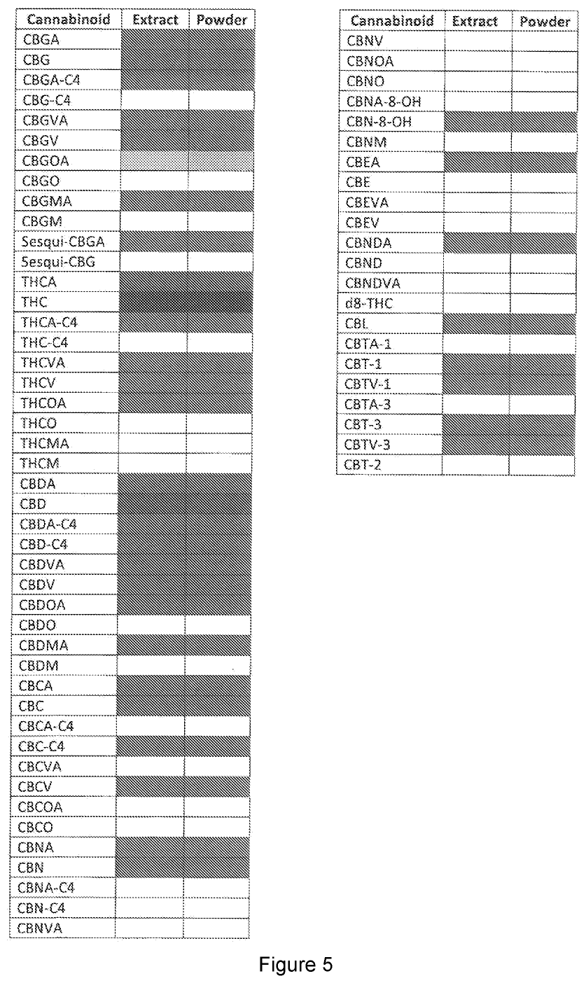

The cannabinoids from the Cannabis extract and from the obtained powder were identified and quantified using a UHPLC system and chromatographic method by reverse phase, coupled with a Q Exactive™ Focus Hybrid Quadrupole-Orbitrap MS (Thermo Scientific, Bremen, Germany). FIG. 5 shows the heatmap of the cannabinoids as cannabinoid concentration (% w/w) normalized to the sum of all cannabinoids in the sample. The darker the shade, the higher the cannabinoid concentration. Comparison of the shades of the cannabinoid concentrations of Cannabis extract vs. the composition of the present invention (powder) revealed substantially identical heatmaps indicating the preservation of the cannabinoid fingerprint in the composition.

D. Composition with Sulfobutylether β-Cyclodextrin Carrier:

0.321 gram of the Cannabis extract was dissolved in 10 ml 100% ethanol as a first liquid solvent at room temperature in a round-bottomed flask yielding a first cannabinoid solution. 3.85 gram of sulfobutylether β-cyclodextrin was dissolved in 5 ml water as a second liquid solvent yielding a second cyclodextrin solution. The cyclodextrin solution was added dropwise to the cannabinoid solution with vigorous stirring using a magnetic stirrer at room temperature. Ethanol was removed from the flask under reduced pressure (water aspirator) at 40° C. using a rotary evaporator followed by water removal using freeze-drying. After freeze-drying, a flowable light-yellow powder remained in the flask.

E. Composition with Lactose Carrier

0.347 gram of the Cannabis extract was dissolved in 12 ml 100% ethanol as a first liquid solvent at room temperature in a round-bottomed flask yielding a first cannabinoid solution. While the first solution was being stirred with a magnetic stirrer at room temperature, 1.024 gram of α-lactose monohydrate was added to the round-bottomed flask. The lactose was observed to form a suspension and swirled around with the stirring. Ethanol was removed from the flask under reduced pressure (water aspirator) at 40° C. using a rotary evaporator. As the ethanol evaporated, it was observed that the lactose remained suspended in the remaining ethanol. After rigorous drying with the rotary evaporator, a dry flaky residue was left on the walls. The residue was easily pushed off the walls of the flask and poured into a porcelain mortar. With minimal grinding using a pestle, the flakes were milled to a flowable brown powder.

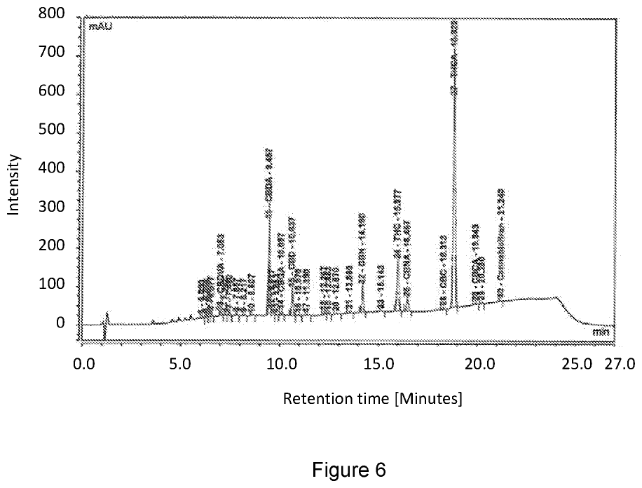

Analytical results substantially identical to those of the cyclodextrin carriers were achieved. FIG. 6 (HPLC-UV trace of the composition with lactose carrier) shows the characteristic cannabinoid fingerprint of the specific Cannabis sativa variety substantially identical to the cannabinoid fingerprint of the Cannabis extract. The peaks identified in the HPLC-UV trace are listed in Table 2 below:

TABLE 2
List of peaks

| | | | | | | Real |
|---|---|---|---|---|---|---|
| Peak | | RT | Area | Amount | w/w | Area |
| No. | Identity | [min] | [mAU*min] | [μg/ml] | [%] | [%] |
| 1 | | 6.090 | 0.400 | NA | NA | 0.30 |
| 2 | | 6.313 | 0.440 | NA | NA | 0.33 |
| 3 | | 6.433 | 0.126 | NA | NA | 0.09 |
| 4 | | 6.577 | 0.647 | NA | NA | 0.49 |
| 5 | CBDVA | 7.053 | 1.395 | 5.3803 | 0.05 | 1.05 |
| 6 | | 7.293 | 0.172 | NA | NA | 0.13 |
| 7 | | 7.480 | 0.835 | NA | NA | 0.63 |
| 8 | | 7.857 | 0.291 | NA | NA | 0.22 |
| 9 | | 8.217 | 0.312 | NA | NA | 0.24 |
| 10 | | 8.607 | 0.175 | NA | NA | 0.13 |
| 11 | CBDA | 9.457 | 19.637 | 98.7195 | 0.99 | 14.78 |
| 12 | | 9.617 | 0.492 | NA | NA | 0.37 |
| 13 | | 9.853 | 0.210 | NA | NA | 0.16 |
| 14 | CBGA | 10.097 | 0.938 | 4.0231 | 0.04 | 0.71 |
| 15 | CBD | 10.637 | 6.864 | 41.3685 | 0.41 | 5.17 |
| 16 | | 10.970 | 0.507 | NA | NA | 0.38 |
| 17 | | 11.390 | 0.394 | NA | NA | 0.30 |
| 18 | | 12.267 | 0.707 | NA | NA | 0.53 |
| 19 | | 12.483 | 0.758 | NA | NA | 0.57 |
| 20 | | 12.870 | 0.419 | NA | NA | 0.32 |
| 21 | | 13.560 | 0.608 | NA | NA | 0.46 |
| 22 | CBN | 14.190 | 6.458 | 20.4826 | 0.20 | 4.86 |
| 23 | | 15.143 | 0.185 | NA | NA | 0.14 |
| 24 | THC | 15.977 | 14.314 | 93.6360 | 0.94 | 10.77 |
| 25 | CBNA | 16.467 | 3.908 | 24.0636 | 0.24 | 2.94 |
| 26 | CBC | 18.313 | 0.443 | 2.4535 | 0.02 | 0.33 |
| 27 | THCA | 18.820 | 70.437 | 401.0407 | 4.01 | 53.01 |
| 28 | CBCA | 19.943 | 0.355 | 5.6299 | 0.06 | 0.27 |
| 29 | | 20.250 | 0.283 | NA | NA | 0.21 |
| 30 | Cannabicitran | 21.240 | 0.152 | 0.7565 | 0.01 | 0.11 |

All of the prepared cannabinoid-carrier compositions that were obtained were flowable powders that provided a faithful representation of the cannabinoid content of the original extract.

F. Composition with Mannitol Carrier:

0.450 gram of the Cannabis extract is dissolved in 15 ml 100% ethanol as a first liquid solvent at room temperature in a round-bottomed flask yielding a first cannabinoid solution. While the first solution is being stirred with a magnetic stirrer at room temperature, 2.25 gram of D-mannitol is added to the round-bottomed flask to form a suspension. Ethanol is then removed from the flask under reduced pressure (water aspirator) at 40° C. using a rotary evaporator to yield a composition containing a plurality of cannabinoids and D-mannitol.

G. Composition with Mesoporous Silica Carrier:

0.5 gram of the Cannabis extract is dissolved in 100% ethanol as a first liquid solvent at room temperature in a round-bottomed flask yielding a first cannabinoid solution. While the first solution is being stirred with a magnetic stirrer at room temperature, 3 grams of mesoporous silica are added to the round-bottomed flask to form a suspension. Ethanol is then removed from the flask under reduced pressure (water aspirator) at 40° C. using a rotary evaporator to yield a composition containing a plurality of cannabinoids and mesoporous silica.

### Micronization

Samples of cannabinoid-bearing powders with a methyl-β-cyclodextrin carrier and a lactose carrier were separately fed into a commercially-available vortex mill (Superfine Ltd., Kidmat Galil, Israel). In both cases, the resulting comminuted powders were flowable. In both cases, the particles had a homogeneous appearance when studied under a microscope, showing no evidence of oil release.

Both samples were analyzed using a commercially-available particle-size analyzer by Malvern Panalytical Ltd. (Malvern, UK), showing that micron-sized particles were achieved with a relatively narrow particle-size distribution.

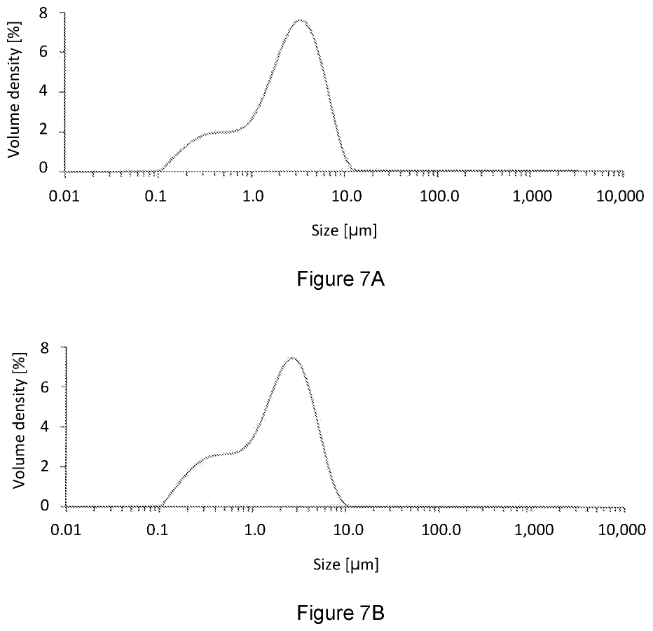

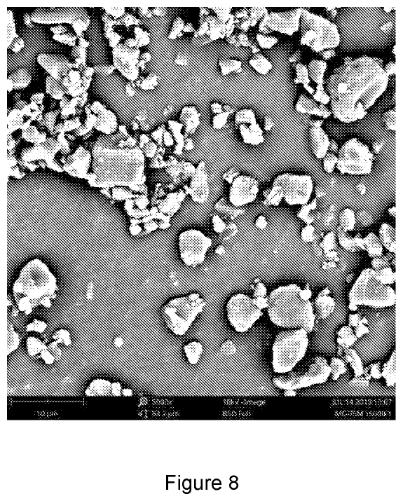

FIG. 7A shows the particle size distribution of a composition with the methyl-β-cyclodextrin carrier subsequent to comminution in a vortex mill, and FIG. 7B shows the particle size distribution of a composition with a lactose carrier subsequent to comminution in a vortex mill. Notably in both cases, more than 99.5% by volume of the comminuted powder had a particle size of less than 10 micrometers. FIG. 8 shows a Scanning Electron Microscope image of micronized powder according to embodiments of the present invention obtained using methyl-β-cyclodextrin carrier. The image indicates the presence of distinct particles constituting the powder. It was apparent that the resulting powders could be size separated to provide fractions having even narrower size distributions, e.g., using a cyclonic separator.

### Preparation of a Solid Terpene Composition in the Form of a Powder

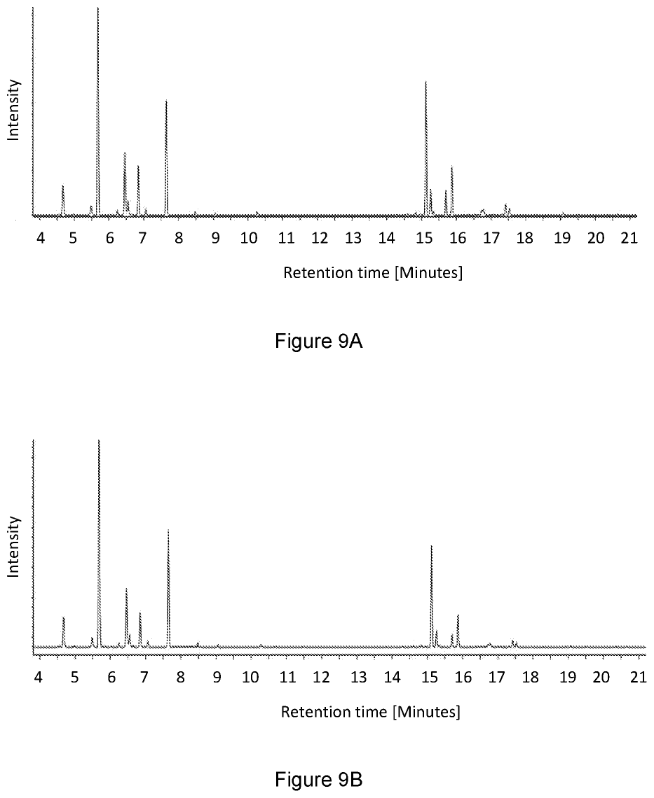

0.340 gram of the terpene containing extract was dissolved in 2 ml ethanol (100%) to provide a terpene solution. 3.4 gram of hydroxypropyl-β-cyclodextrin was dissolved in 1 ml water to provide a cyclodextrin solution. The cyclodextrin solution was added dropwise to the terpene solution with vigorous stirring using a magnetic stirrer at room temperature. Subsequently, the solvents were removed from the flask by freeze-drying. After freeze-drying, a flowable light-yellow powder remained in the flask. FIG. 9B shows that the HPLC-UV trace of the obtained composition has similar characteristic terpene constituents and relative intensities as the terpene containing extract (FIG. 9A).

### Pharmacokinetic Study

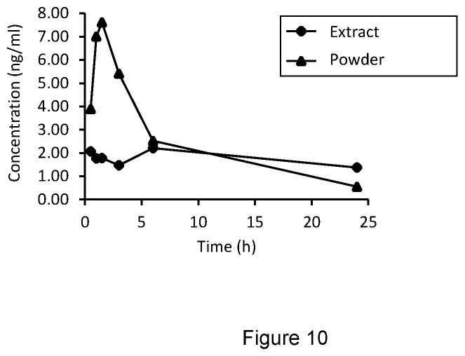

Comparative PK study of Cannabis extract versus the powder of the present invention was conducted as follows: 2 groups, each containing 3 rats were used. A powder according to one embodiment of the present invention was orally administered to one group using the gavage technique at a calculated dosage of 50 mg cannabinoids/kg. The second group was administered with a Cannabis extract at the same dosage and administration route. CBD blood concentrations were tested at 0.5, 1, 1.5, 3, 6, and 24 hours after administration. The results in ng/ml are shown in Table 3 and FIG. 10.

TABLE 3
CBD blood concentrations:

| | | A powder according |
|---|---|---|
| | Cannabis | to embodiments of |
| Time | extract | the present invention |
| 0.5 h | 2.07 | 3.88 |
| 1 h | 1.76 | 7.00 |
| 1.5 h | 1.78 | 7.62 |
| 3 h | 1.47 | 5.41 |
| 6 h | 2.21 | 2.52 |
| 24 h | 1.37 | 0.55 |

The results of THC, THCA, CBDA, CBG, and CBGA blood concentrations in ng/ml are summarized in Tables 4-8, respectively, below:

TABLE 4
THC blood concentrations:

| | | A powder according |
|---|---|---|
| | Cannabis | to embodiments of |
| Time | extract | the present invention |
| 0.5 h | 1.993 | 8.588 |
| 1 h | 2.450 | 34.232 |
| 1.5 h | 4.771 | 22.599 |
| 3 h | 3.022 | 30.108 |
| 6 h | 6.442 | 17.519 |
| 24 h | 2.736 | 2.600 |

TABLE 5
THCA blood concentrations:

| | | A powder according |
|---|---|---|
| | Cannabis | to embodiments of |
| Time | extract | the present invention |
| 0.5 h | 37.057 | 72.387 |
| 1 h | 50.075 | 127.578 |
| 1.5 h | 25.104 | 102.747 |
| 3 h | 14.015 | 102.060 |
| 6 h | 17.182 | 21.116 |
| 24 h | 3.996 | 2.713 |

TABLE 6
CBDA blood concentrations:

| | | A powder according |
|---|---|---|
| | Cannabis | to embodiments of |
| Time | extract | the present invention |
| 0.5 h | 15.504 | 52.545 |
| 1 h | 22.172 | 76.475 |
| 1.5 h | 12.005 | 78.915 |
| 3 h | 7.049 | 56.334 |
| 6 h | 8.986 | 6.499 |
| 24 h | 2.154 | 1.295 |

TABLE 7
CGB blood concentrations:

| | | A powder according |
|---|---|---|
| | Cannabis | to embodiments of |
| Time | extract | the present invention |
| 0.5 h | 0.576 | 1.704 |
| 1 h | 0.940 | 3.803 |
| 1.5 h | 0.632 | 3.049 |
| 3 h | 0.814 | 3.372 |
| 6 h | 0.905 | 1.868 |
| 24 h | 0.843 | 1.165 |

TABLE 8
CBGA blood concentrations:

| | | A powder according |
|---|---|---|
| | Cannabis | to embodiments of |
| Time | extract | the present invention |
| 0.5 h | 23.816 | 115.133 |
| 1 h | 62.116 | 221.077 |
| 1.5 h | 39.266 | 177.219 |
| 3 h | 49.116 | 230.039 |
| 6 h | 65.268 | 122.669 |
| 24 h | 44.984 | 67.468 |

Thus, the results indicate a significant increase in absorption of CBD, THC, THCA, CBDA, CBG, and CBGA from the solid cannabinoid powder composition of the present invention as compared to the Cannabis extract.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method for preparing a solid cannabinoid composition in the form of a powder, the method comprising the steps of:
(i) providing a first solution comprising an extract of Cannabis comprising a plurality of cannabinoids dissolved in a first solvent, wherein said extract, when dried to a residual solvent of about 5,000 parts per million (ppm) or less, is a non-powder crude oil having a viscosity of at least 3,000 cps as determined at 40° C.;
(ii) adding a carrier to the first solution to obtain a mixture comprising said plurality of cannabinoids and carrier; and
(iii) removing at least 90% by weight of the solvent from the mixture, thereby obtaining a solid cannabinoid composition in the form of a powder,
wherein the plurality of cannabinoids in the solid cannabinoid composition has a chemical signature which is substantially identical to the chemical signature of the plurality of cannabinoids in the extract of Cannabis, and wherein the solid cannabinoid composition has a Hausner ratio of less than 1.59.

2. The method of claim 1, wherein the carrier comprises a modified or unmodified cyclodextrin selected from: α-cyclodextrin, β-cyclodextrin, γ-cyclodextrin, 2-hydroxypropyl-β-cyclodextrin, methyl-β-cyclodextrin, and a mixture or combination thereof.

3. (canceled)

4. The method of claim 1, wherein the carrier comprises a sugar selected from: sucrose, dextrose, molasses, lactose, and a mixture or combination thereof.

5. (canceled)

6. The method of claim 1, wherein the carrier comprises a sugar alcohol selected from: mannitol, sorbitol, maltitol, xylitol, arabitol, isomalt, erythritol, glycerol, lactitol, and a mixture or combination thereof.

7. The method of claim 1, wherein the weight percent ratio of the plurality of cannabinoids to the carrier is in the range of about 1:1 to about 1:20.

8. (canceled)

9. The method of claim 1, wherein the viscosity of said dried extract of Cannabis is in the range of about 3,000 to about 2,000,000 cps as determined at 40° C.

10. The method of claim 1, wherein said extract is obtained from a Cannabaceae species selected from: Cannabis sativa, Cannabis indica, Cannabis ruderalis, and a mixture or combination thereof.

11. The method of claim 10, wherein said extract is obtained from a single species of Cannabis.

12. The method of claim 1, wherein said extract is obtained using at least one of: organic solvent extraction, carbon dioxide (dry ice) extraction, supercritical and subcritical carbon dioxide extraction, hydrocarbon extraction, rosin press, and a combination thereof.

13. The method of claim 1, wherein the first solvent is an organic solvent selected from the group consisting of: C₁-C₈ aliphatic alcohols, C₁-C₁₀ aliphatic hydrocarbons, C₆-C₁₀ aromatic hydrocarbons, C₂-C₈ aliphatic esters, C₂-C₈ aliphatic ketones, C₄-C₈ ethers, C₁-C₁₀ halo-substituted aliphatic hydrocarbons, C₂-C₈ aliphatic amides, and a mixture or combination thereof; or the first solvent is selected from the group consisting of: ethanol, methanol, isopropyl alcohol, n-butanol, t-butyl alcohol, acetone, methyl ethyl ketone, toluene, benzene, hexane, cyclohexane, heptane, pentane, methyl acetate, ethyl acetate, t-butyl acetate, isopropyl acetate, diisopropyl ether, methyl t-butyl ether, tetrahydrofuran, dioxane, dichloromethane, dichloroethane, chloroform, carbon tetrachloride, tetrachloroethylene, tetrachloroethane, dimethylformamide, dimethylacetamide, and a mixture or combination thereof.

14. (canceled)

15. The method of claim 13, wherein the first solvent is ethanol.

16. The method of claim 1, wherein the carrier is dissolved or suspended in a second solvent prior to step (ii).

17. The method of claim 16, wherein the second solvent is miscible in the first solvent; or the second solvent is an aqueous solvent.

18. (canceled)

19. The method of claim 16, wherein the ratio between the first solvent and the second solvent is in the range of about 1:5 to about 1:1.

20. The method of claim 1, wherein step (iii) is performed using at least one of the following techniques: evaporation, freeze-drying (lyophilization), distillation, air drying, spray drying, fluid bed drying, or a combination thereof.

21. The method of claim 1, wherein the plurality of cannabinoids are selected from the group consisting of: cannabidivarinic acid (CBDVA), cannabidiolic acid (CBDA), cannabigerolic acid (CBGA), cannabidiol (CBD), cannabinol (CBN), cannabinolic acid (CBNA), tetrahydrocannabinol (THC), cannabichromene (CBC), cannabichromenic acid (CBCA), tetrahydrocannabinolic acid (THCA), cannabicitran, and a mixture or combination thereof; optionally the plurality of cannabinoids further comprises at least one of: tetrahydrocannabivarin (THCV), cannabigerol (CBG), sesquicannabigerol (sesqui-CBG), sesquicannabigerolic acid (sesqui-CBGA), CBGA-C4, CBG-C4, cannabigerovarinic acid (CBGVA), cannabigerivarin (CBGV), cannabigerorcinic acid (CBGOA), cannabigerorcin (CBGO), cannabigerolic acid monomethyl ether (CBGMA), cannabigerol monomethyl ether (CBGM), cannabicyclol (CBL), cannabicyclolic acid (CBLA), THCA-C4, THC-C4, tetrahydrocannabivarin carboxylic acid (THCVA), tetrahydrocannabivarin (THCV), tetrahydrocannabiorcolic acid (THCOA), tetrahydrocannabiorcol (THCO), THCMA, THCM, CBDA-C4, CBD-C4, cannabidiorcolic acid (CBDOA), cannabidiorcol (CBDO), cannabidiolic acid monomethyl ether (CBDMA), cannabidiol monomethylether (CBDM), cannabichromenic acid (CBCA), cannabichromene (CBC), cannabichromevarinic acid (CBCVA), cannabichromevarin (CBCV), cannabiorchromenic acid (CBCOA), cannabiorchromene (CBCO), cannabinolic acid (CBNA), cannabinol (CBN), cannabinol-C4 (CBN-C4), cannabivarinic acid (CBNVA), cannabivarin (CBNV), cannabiorcolic acid (CBNOA), cannabiorcol (CBNO), CBNA-8-OH, CBN-8-OH, cannabinol methylether (CBNM), cannabielsoin acid (CBEA), cannabielsoin (CBE), cannabielsoic acid (CBEVA), cannabielsoin (CBEV), cannabinodiolic acid (CBNDA), cannabinodiol (CBND), cannabinodivarinic acid (CBNDVA), (−)-Δ⁸-trans-tetrahydrocannabinol (Δ⁸-THC), cannabitriol-1 (CBT-1), CBT-2, CBT-3, CBTA-1, CBTA-3, cannabitriolvarin (CBTV), CBTV-3, and a mixture or combination thereof.

22. (canceled)

23. The method of claim 1, wherein the composition further comprises other components extracted from Cannabis comprising at least one of terpenes, terpenoids, flavonoids, nitrogenous compounds, amino acids, proteins, glycoproteins, sugars, hydrocarbons, fatty acids, esters, lactones, steroids, non-cannabinoid phenols, and a mixture or combination thereof.

24. A solid cannabinoid composition obtained by the method of claim 1.

25. The solid cannabinoid composition of claim 24 for use in therapy; or for use as an adjuvant in a dosage form comprising a non-cannabinoid active pharmaceutical ingredient.

26. (canceled)

27. The solid cannabinoid composition of claim 25, which is in a dosage form selected from: tablet, pill, capsule, pellets, granules, powder, lozenge, sachet, cachet, elixir, suspension, dispersion, emulsion, solution, syrup, aerosol, gel, ointment, lotion, cream, and suppository; or which is adapted for administration via a route selected from: oral, subcutaneous, intratracheal, intrabronchial, intra-alveolar, intraperitoneal, rectal, intravenous, intra-arterial, transdermal, intramuscular, topical, and intranasal; or which is adapted for use in a dry-powder inhaler; or which is filled in a capsule for oral administration.

28-30. (canceled)
